(19) United States
(12) Patent Application Publication
(10) Pub. No.: US 2002/0169126 A1
(43) Pub. Date: Nov. 14, 2002

(54) Compositions and methods for inactivating the Akt oncogene and/or activating the p38 pro-apoptotic gene

(76) Inventors: Mien-Chie Hung, Houston, TX (US); Yong Liao, Houston, TX (US)

Correspondence Address: FULBRIGHT & JAWORSKI L.L.P., SUITE 2400, 600 CONGRESS AVENUE, AUSTIN, TX, 78701, US

(21) Appl. No.: 10/103,542

(22) Filed: Mar. 21, 2002

Related U.S. Application Data
(60) Provisional application No. 60/277,788, filed on Mar. 21, 2001.

(51) Int. Cl. A61K 48/00; A61K 38/17

(52) U.S. Cl. 514/12; 514/44; 424/93.2; 435/320.1

(57) ABSTRACT

The invention provides E1A gene based anti-cancer therapy that targets cancers and tumors involving phospho-Akt expression (and/or Akt activation) and/or phospho-p38 downregulation (and/or p38 inactivation).

The government owns rights in the present invention pursuant to grant number R01-CA58880 from the National Institutes of Health and R01-CA77858 from the MDACC Breast Cancer Research Program.

### BACKGROUND OF THE INVENTION

The present application claims priority to co-pending U.S. Patent Application Serial No. 60/277,788, filed Mar. 21, 2001. The entire text of the above-referenced disclosures is specifically incorporated by reference herein without disclaimer.

1. Field of the Invention

The present invention relates generally to the fields of molecular biology, oncology and gene therapy. More particularly, it concerns the development of E1A gene based anti-cancer therapy that targets the Akt oncogene and/or the p38 pro-apoptotic gene. The invention provides gene therapy for cancers involving phospho-Akt expression and/or phospho-p38 downregulation.

2. Description of Related Art

Cancer has become one of the leading causes of death in the western world, second only behind heart disease. Current estimates project that one person in three in the U.S. will develop cancer, and that one person in five will die from cancer. Major challenges remain to be overcome for all cancers and this makes it essential to uncover the different molecular processes that lead to cancer.

Cancer is a result of multistep molecular processes that transform normal cells into cancerous cells. Molecular models supporting this hypothesis were provided by studies on two DNA tumor viruses, adenovirus and polyomavirus. In the case of adenovirus, it was found that transformation of primary cells required the expression of both the early region 1A (E1A ) and 1B (E1B) genes (Hoveling et al., 1980). Of these, the E1A gene products cooperate with the SV40 middle T antigen or with an activated H-ras gene to transform primary cells (Ruley, 1985).

During the last decade, a number of human malignancies have been correlated with the presence and expression of “oncogenes” in the human genome. More than twenty different oncogenes have now been implicated in tumorigenesis, and are thought to play a direct role in human cancer (Weinberg, 1985). Many of these oncogenes evolve through mutagenesis of a normal cellular counterpart, termed a “proto-oncogene”, which leads to either an altered expression or activity of the gene product. There is considerable data linking proto-oncogenes to cell growth, including their expression during embryonic development (Muller et al., 1982), and in response to certain proliferation signals (Campisi et al., 1983). Moreover, a number of the proto-oncogenes are related to either a growth factor or a growth factor receptor. These observations suggested the involvement of multiple functions in the transformation process, and that various oncogenes may express similar functions on a cellular level.

The adenovirus E1A gene codes for several related proteins which possess a number of interesting properties. E1A can not only complement a second oncogene (H-ras) in transformation but another closely related function allows E1A to immortalize primary cells (Ruley, 1985). For example, introduction of E1A gene products into primary cells has been shown to provide these cells with an unlimited proliferative capacity when cultured in the presence of serum. Another interesting mode of function of E1A is by “trans-activation”, wherein E1A gene products stimulate transcription from a variety of viral and cellular promoters, including the adenovirus early and major late promoter. However, trans-activation is not universal for all promoters. In some instances, E1A causes a decrease in transcription from cellular promoters that are linked to enhancer elements (Haley et al., 1984). It has been shown that exogenously added E1A gene can reduce the metastatic potential of ras-transformed rat embryo fibroblast cells by activating the cellular NM23 gene that is associated with a lower metastatic potential (Pozzatti et al., 1988; Wallich et al., 1985).

Another viral oncoprotein, the SV40 large T antigen (LT) shares structural and functional homology to E1A and c-myc (Figge et al., 1988). LT, E1A and c-myc have transforming domains which share amino acid sequence homology and similar secondary structure (Figge et al., 1988). All three proteins complex with the tumor suppressor, retinoblastoma gene product (Rb) (Whyte et al., 1988, DeCaprio et al., 1988, Rustgi et al., 1991), and the Rb binding domains of LT and E1A coincide with their transforming domains. Based on this similarity, it is believed that LT and E1A transform cells by binding cellular Rb and abrogating its tumor suppressor function. LT, E1A and c-myc are also grouped as immortalization oncogenes as determined by an oncogene cooperation assay using rat embryo fibroblasts (see Weinberg, 1985).

Despite advances in identifying some of the components which contribute to the development of malignancies, it is clear that the art still lacks effective means of suppressing carcinogenesis. Work by Hung and collaborators has established that the E1A gene can in fact suppress transformation, tumorigenicity and metastasis in a variety of cancers (see, e.g., Yu et al. 1991, 1992 and 1993; and the reviews by Hung et al., 1995, Yu and Hung, 1995, and Mymryk, 1996). In addition, Hung and collaborators have made advances in the suppression of oncogenic transformation. Some of these advances, especially in the case of tumors mediated by over-expression of an oncogene variously referred to as c-erbB-2, HBER-2 or neu (referred to herein as the neu oncogene), are described in U.S. Pat. Nos. 5,814,315, 5,651,964, 5,641,484, and 5,643,567, the entire texts of each being specifically incorporated herein by reference. U.S. Pat. No. 6,197,794, incorporated herein by reference, also by Hung and collaborators, describes the construction of a mini-E1A gene, which comprises only essential regions of the E1A gene required for the anti-cancer effects, to provide anti-cancer therapies. Additionally, sensitization of tumor cells to chemo- and radiation-therapy by introducing E1A genes is described in U.S. Pat. No. 5,776,743, to Frisch.

These patents establish the function of E1A as a tumor suppressor gene and further suggest that E1A is a potential therapeutic reagent for the treatment of a variety of human cancers. Indeed, success with the use of E1A as a tumor suppressor gene in animal models of human cancer has merited the initiation of Phase I human clinical trials for multiple indications which are currently being sponsored by Targeted Genetics Corporation at the Virginia Mason Medical Center in Seattle, at the M. D. Anderson Cancer Center in Houston, and at Wayne State University in Detroit, at the Rush Presbyterian St. Luke's Medical Center in Chicago. In addition, Targeted Genetics' European partner, Groupe Fournier, has received approval by the Ministry of Health to begin corresponding clinical trials in France.

In spite of these advances, there are several cancers that involve other oncogenes and signaling molecules that have not been adequately addressed from a treatment standpoint. For example, one class of cancers are those where the Akt oncogene is overexpressed. Akt (also known as PKBγ and RAC-PKγ) is a member of the AKT/PKB family of serine/threonine kinases first isolated from a rat brain cDNA and is expressed predominantly in the central nervous system and the testis (Konishi et al., 1995).

Akt, like other members of the AKT/PKB family, is located in the cytosol of unstimulated cells and translocates to the plasma membrane following stimulation by several ligands including mitogens and survival factors (Meier et al., 1997). This activation is through PI3 kinase which is wortmannin sensitive (Franke et al., 1997). Akt binds to the lipid products of P13 kinase allowing presentation of Akt to its upstream activators by directing its translocation to the membrane via a pleckstrin homology domain (PH) within the protein. Phosphorylation of Akt is necessary for its activation and the kinase responsible for this activation has been identified as PDK1 (Cohen et al., 1997). Once localized to the membrane, Akt mediates several functions within the cell including the metabolic effects of insulin (Walker et al., 1998). Akt activating (for example, enhanced phospho-Akt expressing) cancers are difficult to treat and include ovarian, breast, prostate, brain, colon and other cancers.

Another group of cancers that are largely unaddressed in the art are those where the proapoptotic factor p38 is found to be downregulated. p38, a serine-threonine kinase protein, is also a mitogen-activated protein kinase (MAPK). Phosphorylation of p38 is necessary for its activation. p38 is also known as cytokine suppressive anti-inflammatory drug binding protein (CSBP) and RK. p38 was initially isolated from murine pre-B cells that were transfected with the lipopolysaccharide (LPS) receptor CD14 and induced with LPS. p38 has since been isolated and sequenced, as has the cDNA encoding it in humans and mouse.

p38 has been identified as an early stress responsive MAPK in relation to events that damage DNA. Activation of p38 has been observed in cells stimulated by stresses, such as treatment of lipopolysaccharides (LPS), UV, anisomycin, or osmotic shock, and by cytokines, such as IL-1 and TNF.

p38 MAPK has also been shown to be a prominent activator of p53 both in response to UV radiation as well as certain anticancer-drugs. p38 physically binds and phosphorylates p53 at critical serine residues. This is associated with p53 mediated transcriptional cascades that ultimately lead to apoptosis. Thus, p38 is a pro-apoptotic factor, that causes apoptosis in cells with damaged DNA.

Although the role of E1A has been described in the molecular pathways that cause certain types of cancers, such as neu-mediated cancers, there is still a need to systematically investigate and identify the other genes and proteins that are regulated by E1A which may be involved in the development and progression of other types of cancers. Neither the Hung patents or the Frisch patents outline the role of Akt and/or p38 in various cancers and further no specific therapies directed to these cancers have been addressed. Therefore, there is as yet no particularly successful way of suppressing cancers associated with activation of the Akt oncogene (e.g., enhanced phospho-Akt expression) and/or those involving the inactivation of p38 (e.g., downregulation of phospho-p38) or both. Hence, there is a need for methods of treatment for these types of cancers.

### SUMMARY OF THE INVENTION

The present invention provides E1A based gene-therapy to cancers that have either upregulated phosphorylation levels of an oncogene, Akt, or downregulated phosphorylation levels of a pro-apoptotic gene product, p38, or both. Examples of such cancers, include but are not limited to, breast cancers, colon cancers, pancreatic cancers, ovarian cancers, colorectal cancers, large cell lymphomas, neuroblastomas, etc.

The instant invention relates to the treatment of cancer via the use of E1A, as defined herein, to, 1) inactivate or downregulate Akt activity; or 2) activate or upregulate p38 activity; or 3) both inactivate or downregulate Akt activity and activate or upregulate p38 activity. In cases where both Akt and p38 activities are affected, Akt activity can be inactivated or downregulated either prior to or after the activation or upregulation of p38 activity or alternatively, it is possible that both Akt activity inactivation or downregulation and p38 activity activation or upregulation can occur simultaneously.

The inventors have for the first time demonstrated the modulation of these targets by the E1A gene products exemplified here by an E1A protein, peptide, or polypeptide or a nucleic acid encoding the E1A protein, peptide, or polypeptide.

Therefore, methods of inactivating Akt in a cell comprising contacting the cell with an E1A protein, peptide, or polypeptide are provided. In some embodiments, the cell is a cell that overexpresses activated Akt. Activated Akt is the phosphorylated form of the Akt oncogene. In other embodiments, the cell is a cancer cell. In specific embodiments, the cell is a breast cancer cell, ovarian cancer cell, pancreatic cancer cell, prostate cancer cell, colon cancer cell, brain cancer cell, or a rectal cancer cell.

In other embodiments, the cell is comprised in vitro. In yet other embodiments, the cancer cell is comprised in a tumor. In still other embodiments, the cell is comprised in an animal. In specific aspects the animal is human.

In some embodiments the methods are defined as methods of treating cancer. In other embodiments, the methods are defined as methods for preventing cancer. In some aspects the transformation of the cell is suppressed. In other aspects the growth of the cell is suppressed. The growth of the cell may be metastatic growth.

In other embodiments, the cell is characterized by p38 inactivation. Activated p38 comprises the phosphorylated form of the p38 pro-apoptotic protein. Thus, inactivated p38 corresponds to the non-phosphorylated forms of p38. In some embodiments, contacting the E1A protein, peptide, or polypeptide results in p38 activation.

In yet other embodiments, contacting the cell with the E1A protein, peptide, or polypeptide comprises providing a nucleic acid encoding the E1A protein, peptide, or polypeptide and expressing the E1A protein, peptide, or polypeptide. The expression of E1A protein, peptide, or polypeptide occurs in the cell.

In some aspects, the E1A protein, peptide, or polypeptide is encoded by a nucleic acid vector. In specific aspects, the vector is a viral vector and may be an adenoviral vector, a retroviral vector or a lentiviral vector.

In other specific aspects, the E1A protein, peptide, or polypeptide or a nucleic acid encoding the E1A protein, peptide, or polypeptide is comprised in a liposome. The liposome may be comprised of any lipid-related compound, however in some specific embodiments the liopsome is comprised of DOTMA, DOPE, or DC-Chol. In other specific embodiments, the liposome comprises DC-Chol. In yet other specific embodiments, the liposome comprises both DC-Chol and DOPE.

In some aspects, the contacting comprises administering the E1A protein, peptide, or polypeptide or a nucleic acid encoding the E1A protein, peptide, or polypeptide to an animal. In such aspects, the E1A protein, peptide, or polypeptide or nucleic acid encoding the E1A protein, peptide, or polypeptide may be administered locally. For example, the E1A protein, peptide, or polypeptide or nucleic acid encoding the E1A protein, peptide, or polypeptide may be administered by direct intratumoral injection or by injection into tumor vasculature.

Alternatively, the E1A protein, peptide, or polypeptide or nucleic acid encoding the E1A protein, peptide, or polypeptide may be administered systemically. For example, one may administer the E1A protein, peptide, or polypeptide or nucleic acid encoding the E1A protein, peptide, or polypeptide intravenously, intra-arterially, intra-peritoneally.

The inventors also envision combination therapies for the treatment of cancers in conjunction with the present invention. Therefore, in some embodiments the methods further comprise contacting the cell with a chemotherapeutic agent. The chemotherapeutic agent can be gemcitabine, novelbine, taxtere, taxol, cisplatin, doxorubicin, VP16, TNF, emodin, daunorubicin, dactinomycin, mitoxantrone, procarbazine, mitomycin, carboplatin, bleomycin, etoposide, teniposide, mechlroethamine, cyclophosphamide, ifosfamide, melphalan, chlorambucil, ifosfamide, melphalan, hexamethylmelamine, thiopeta, busulfan, carmustine, lomustine, semustine, streptozocin, dacarbazine, adriamycin, 5-fluorouracil (5FU), camptothecin, actinomycin-D, hydrogen peroxide, nitrosurea, plicomycin, tamoxifen, transplatinum, vincristin, vinblastin, TRAIL, or methotrexate.

The E1A protein, peptide, or polypeptide or nucleic acid encoding the E1A protein, peptide, or polypeptide and the chemotherapeutic agent may be administered at the same time or alternatively, may be administered at different times. For example, the E1A protein, peptide, or polypeptide or nucleic acid encoding the E1A protein, peptide, or polypeptide may be administered before administration of the chemotherapeutic agent or after administration of the chemotherapeutic agent.

The invention also provides methods of activating p38 in a cell comprising contacting the cell with an E1A protein, peptide, or polypeptide. In some embodiments, the cell is a cell that underexpresses activated p38. In other embodiments, the cell is a cancer cell. In some specific embodiments, the cancer cell is a breast cancer cell, an ovarian cancer cell, a pancreatic cancer cell, a prostate cancer cell, or a colon cancer cell.

In some embodiments, the cell is comprised in vitro. In other embodiments, the cancer cell is comprised in a tumor. In still other embodiments, the cell is comprised in an animal. In some specific embodiments, the animal is human.

In some embodiments, the method is defined as a method of treating cancer. In other embodiments, the method is defined as a method of preventing cancer. In some aspects, the transformation of the cell is suppressed. In other aspects, the growth of the cell is suppressed. The growth of the cell may be metastatic growth.

In yet other embodiments, the method is further defined as a method of inducing apoptosis.

In some aspects, the cell is further characterized by Akt activation. In such aspects, contacting of the E1A protein, peptide, or polypeptide results in Akt inactivation.

In other embodiments, contacting the cell with the E1A protein, peptide, or polypeptide comprises providing a nucleic acid encoding the E1A protein, peptide, or polypeptide and expressing the E1A protein, peptide, or polypeptide. The expression of E1A protein, peptide, or polypeptide occurs in the cell.

In some aspects, the E1A protein, peptide, or polypeptide is encoded by a nucleic acid vector. In specific aspects, the vector is a viral vector and may be an adenoviral vector, a retroviral vector or a lentiviral vector.

In other specific aspects, the E1A protein, peptide, or polypeptide or a nucleic acid encoding the E1A protein, peptide, or polypeptide is comprised in a liposome. The liposome may be comprised of any lipid-related compound, however in a specific embodiment the liopsome is comprised of DOTMA, DOPE, or DC-Chol. In some specific embodiments, the liposome comprises DC-Chol. In other more specific embodiments, the liposome comprises both DC-Chol and DOPE.

In some aspects, the contacting comprises administering the E1A protein, peptide, or polypeptide or a nucleic acid encoding the E1A protein, peptide, or polypeptide to an animal. In such aspects, the E1A protein, peptide, or polypeptide or nucleic acid encoding the E1A protein, peptide, or polypeptide may be administered locally. For example, the E1A protein, peptide, or polypeptide or nucleic acid encoding the E1A protein, peptide, or polypeptide may be administered by direct intratumoral injection or by injection into tumor vasculature.

Alternatively, the E1A protein, peptide, or polypeptide or nucleic acid encoding the E1A protein, peptide, or polypeptide may be administered systemically. For example, one may administer the E1A protein, peptide, or polypeptide or nucleic acid encoding the E1A protein, peptide, or polypeptide intravenously, intra-arterially, intra-peritoneally.

The inventors also envision combination therapies for the treatment of cancers in conjunction with the present invention. Therefore, in some embodiments the methods further comprises contacting the cell with a chemotherapeutic agent. The chemotherapeutic agent can be gemcitabine, novelbine, taxtere, taxol, cisplatin, doxorubicin, VP 16, TNF, emodin, daunorubicin, dactinomycin, mitoxantrone, procarbazine, mitomycin, carboplatin, bleomycin, etoposide, teniposide, mechlroethamine, cyclophosphamide, ifosfamide, melphalan, chlorambucil, ifosfamide, melphalan, hexamethylmelamine, thiopeta, busulfan, carmustine, lomustine, semustine, streptozocin, dacarbazine, adriamycin, 5-fluorouracil, camptothecin, actinomycin-D, hydrogen peroxide, nitrosurea, plicomycin, tamoxifen, transplatinum, vincristin, vinblastin, TRAIL, or methotrexate.

The E1A protein, peptide, or polypeptide or nucleic acid encoding the E1A protein, peptide, or polypeptide and the chemotherapeutic agent may be administered at the same time or alternatively, may be administered at different times. For example, the E1A protein, peptide, or polypeptide or nucleic acid encoding the E1A protein, peptide, or polypeptide may be administered before administration of the chemotherapeutic agent or after administration of the chemotherapeutic agent.

The invention also provides methods of downregulating phospho-Akt expression and upregulating phospho-p38 expression in a cell comprising contacting the cell with an E1A protein, peptide, or polypeptide.

In specific embodiments, the cell is a cancer cell and may be a breast cancer cell, an ovarian cancer cell, a pancreatic cancer cell, a prostate cancer cell, a colon cancer cell, a bladder cancer cell, a lung cancer cell, a liver cancer cell, a stomach cancer cell, a testicular cancer cell, a brain cancer cell, a lymphatic cancer cell, a skin cancer cell, a brain cancer cell, a bone cancer cell, a rectal cancer cell, or a blood cancer cell.

In other embodiments, the cell is comprised in vitro. In yet other embodiments, the cancer cell is comprised in a tumor. In still other embodiments, the cell is comprised in an animal. In specific aspects, the animal is human.

In some embodiments the methods are defined as methods of treating cancer. In other embodiments, the methods are defined as methods of preventing cancer. In some aspects, the transformation of the cell is suppressed. In other aspects, the growth of the cell is suppressed. The growth of the cell may be metastatic growth.

In yet other embodiments, the method is further defined as a method of inducing apoptosis.

In other embodiments of the methods, contacting the cell with the E1A protein, peptide, or polypeptide comprises providing a nucleic acid encoding the E1A protein, peptide, or polypeptide and expressing the E1A protein, peptide, or polypeptide. The expression of E1A protein, peptide, or polypeptide occurs in the cell.

In some aspects, the E1A protein, peptide, or polypeptide is encoded by a nucleic acid vector. In specific aspects, the vector is a viral vector and may be an adenoviral vector, a retroviral vector or a lentiviral vector.

In other specific aspects, the E1A protein, peptide, or polypeptide or a nucleic acid encoding the E1A protein, peptide, or polypeptide is comprised in a liposome. The liposome may be comprised of any lipid-related compound, however in some specific embodiments the liposome is comprised of DOTMA, DOPE, or DC-Chol. In other specific embodiments, the liposome comprises DC-Chol. In yet other specific embodiments, the liposome comprises both DC-Chol and DOPE.

In some aspects, the contacting comprises administering the E1A protein, peptide, or polypeptide or a nucleic acid encoding the E1A protein, peptide, or polypeptide to an animal. In such aspects, the E1A protein, peptide, or polypeptide or nucleic acid encoding the E1A protein, peptide, or polypeptide may be administered locally. For example, the E1A protein, peptide, or polypeptide or nucleic acid encoding the E1A protein, peptide, or polypeptide may be administered by direct intratumoral injection or by injection into tumor vasculature.

Alternatively, the E1A protein, peptide, or polypeptide or nucleic acid encoding the E1A protein, peptide, or polypeptide may be administered systemically. For example, one may administer the E1A protein, peptide, or polypeptide or nucleic acid encoding the E1A protein, peptide, or polypeptide intravenously, intra-arterially, intra-peritoneally.

The inventors also envision combination therapies for the treatment of cancers in conjunction with the present invention. Therefore, in some embodiments the method further comprises contacting the cell with a chemotherapeutic agent. The chemotherapeutic agent can be gemcitabine, novelbine, taxtere, taxol, cisplatin, doxorubicin, VP16, TNF, emodin, daunorubicin, dactinomycin, mitoxantrone, procarbazine, mitomycin, carboplatin, bleomycin, etoposide, teniposide, mechlroethamine, cyclophosphamide, ifosfamide, melphalan, chlorambucil, ifosfamide, melphalan, hexamethylmelamine, thiopeta, busulfan, carmustine, lomustine, semustine, streptozocin, dacarbazine, adriamycin, 5-fluorouracil, camptothecin, actinomycin-D, hydrogen peroxide, nitrosurea, plicomycin, tamoxifen, transplatinum, vincristin, vinblastin, TRAIL, or methotrexate.

The E1A protein, peptide, or polypeptide or nucleic acid encoding the E1A protein, peptide, or polypeptide and the chemotherapeutic agent may be administered at the same time or alternatively, may be administered at different times. For example, the E1A protein, peptide, or polypeptide or nucleic acid encoding the E1A protein, peptide, or polypeptide may be administered before administration of the chemotherapeutic agent or after administration of the chemotherapeutic agent.

Also provided are methods of inducing apoptosis in a cell comprising contacting the cell with an E1A protein, peptide, or polypeptide. In some embodiments the cell is an p38 underexpressing cell. The cell may also comprise a cancer cell. In such aspects, the cancer cell may be comprised in a tumor. In still other embodiments, the cell is comprised in an animal. In specific aspects the animal is human.

In some embodiments, contacting the cell with the E1A protein, peptide, or polypeptide comprises providing a nucleic acid encoding the E1A protein, peptide, or polypeptide and expressing the E1A protein, peptide, or polypeptide. In specific aspects, the expression of E1A protein, peptide, or polypeptide occurs in the cell.

In some aspects the E1A protein, peptide, or polypeptide is encoded by a nucleic acid vector. In other embodiments, the E1A protein, peptide, or polypeptide or a nucleic acid encoding the E1A protein, peptide, or polypeptide is comprised in a liposome. In other embodiments, the contacting comprises administering the E1A protein, peptide, or polypeptide or a nucleic acid encoding the E1A protein, peptide, or polypeptide to an animal.

Following longstanding patent law convention, the word “a” and “an”, when used in conjunction with the word comprising, mean “one or more” in this specification, including the claims.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. E1A downregulates Akt and upregulates p38 phosphorylation in breast cancer MDA-MB-231 and MCF-7 cells.

FIG. 2A & FIG. 2B. Akt and p38 are not physically associated but reciprocally regulated.

FIG. 3. Inactivation of Akt or activation of p38 is required for E1A to repress cell growth in vitro.

FIG. 4A & FIG. 4B. E1A represses anchorage-dependent and anchorage-independent cells growth in soft agar in Akt activating breast cancer MDA-MB-231 cells.

FIG. 5. E1A represses tumorigenicity in vivo in Akt activating breast cancer MDA-MB-231 cells.

### DESCRIPTION OF ILLUSTRATIVE EMBODIMENTS

Identification of key genes and processes that lead to the pathology of cancer of will provide novel and specific treatment regimens for cancer therapy. Although some of the mechanisms of E1A function that lead to the anti-cancer effects are known, for example, in neu mediated cancers, there is still a need to identify other molecular players that are regulated by E1A.

The present inventors have found that expression of the E1A gene downregulates the oncogene Akt, which is involved in promoting cell survival and is additionally known to be overexpressed in several cancers. Furthermore, the inventors have also discovered that E1A expression upregulates another protein, p38, which is a cell-death promoter that is also known to be downregulated in several human cancers. Thus, the present invention provides E1A-based anti-cancer therapies for cancers involving either Akt upregulation or p38 downregulation or both. Such cancers are exemplified by, but not limited to, breast, colon, ovarian, pancreatic, brain, and rectal cancers.

The active forms of both the Akt protein and the p38 protein comprise phosphorylated versions of those proteins. Utilizing phospho-specific antibodies against Akt the present inventors showed that levels of phosphorylated Akt (activated Akt), were reduced in the breast cancer cells that were transfected with a construct expressing E1A The E1A expressing construct here is defines as a a nucleic acid encoding and expressing a E1A protein, peptide, or polypeptide. Additionally, phospho-specific antibodies against p38 showed dramatically increased levels of phosphorylated p38 (activated p38), in breast cancer cells transfected with E1A expressing constructs. These in vitro results were further confirmed by in vivo studies using orthotropic mouse breast cancer models with either enhanced Akt and/or decreased p38 activities or both. Therapy with a E1A protein, peptide, or polypeptide or nucleic acid encoding the E1A protein, peptide, or polypeptides decreased the levels of activated Akt (oncogene) and increased levels of activated p38 (pro-apoptotic proetin), thereby repressing the induced tumorigenicity in the nude mice.

The inventors have also performed several experiments that address the mechanisms of action of Akt and/or p38. For example, blockade of p38 activity, by use of a synthetic compound, SB203580, increased the Akt activity in E1A stable expressing MDA-MB-231 cells. Reciprocally, when Akt activity was blocked, using wortmannin, p38 activity was enhanced. Thus, Akt and p38 are reciprocally regulated. Similar results were also seen in experiments that used genetics to block either p38 or Akt activities.

Effects on chemosensitization in cancers involving Akt and/or p38 by E1A were also performed. It was shown that E1A mediates sensitization to chemotherapeutics such as Taxol in addition to downregulating Akt and upregulating p38 activities. These effects of E1A are further additions to the now well known ability of E1A to downregulate the neu oncogene. These experiments were performed in stable human cancer cell lines expressing high levels of wild type E1A proteins in a background of low levels of neu. In vivo studies using an orthotropic mouse cancer model also confirmed the in vitro results that introduction of E1A significantly increased the sensitivity of cancer cells to Taxol cytotoxicity; dramatically enhanced tumor regression; and prolonged survival in mice as compared to controls.

In addition, blocking p38 activity, by using a chemical inhibitor, such as, SB203580, or by a genetic approach, such as, using an inducible dominant negative p38 mutant, and introducing constitutively active Akt partially abrogated the ability of E1A to sensitize cells to Taxol-mediated cytotoxicity. Moreover, introducing p38 or blocking Akt activity in cancer cells also enhanced sensitivity to Taxol induced apoptosis. Although a physical association between Akt and p38 was not detected by immunoprecipitation, cross regulations between these two pathways were observed. Thus, the inventors have demonstrated that E1A-mediated chemosensitization can be achieved by modulating the apoptotic threshold through down-regulation of Akt and up-regulation of p38 activities in breast cancer cells. These results provide a powerful strategy for the adjuvant chemotherapy of human cancers involving Akt downregulation and/or p38 upregulation, by combinations of E1A gene therapy with chemotherapy.

Thus, the present invention provides adenovirus type 5 E1A based gene-therapy for patients with tumors/cancers associated with Akt activation or p38 inactivation.

A. E1A Proteins, Peptides, and Polypeptides

The present invention provides gene therapy by providing one or more E1A gene product(s), defined herein as, an adenovirus E1A protein, peptide, or polypeptide or a nucleic acid encoding the E1A protein, peptide, or polypeptide, to a patient afflicted with a cancer that is characterized by either an increased Akt activity/expression or a decreased p38 activity/expression or both. The E1A protein, peptide, or polypeptide or nucleic acid encoding the E1A protein, peptide, or polypeptides, contemplated as useful in this invention, can encode a variety of proteins. The current discussion describes various specific E1A gene products that may be used for therapy in context of the present invention.

Generally, it will be most convenient to simply use the wild type E1A gene directly. However, it is contemplated that certain regions of the E1A gene may be employed exclusively without employing the entire wild type E1A gene. It is contemplated that it will ultimately be preferable to employ the smallest region needed to suppress the Akt gene and/or the smallest region required to activate the p38 gene, so that one is not introducing unnecessary DNA into cells which receive the E1A gene construct.

In some embodiments, it is contemplated that the 13S product or the 12S product will be used. The E1A gene products referred to as the 13S and the 12S products reflect the sedimentation value of two mRNAs that encode these products. These two mRNAs arise through differential splicing of a common precursor, and code for related proteins of 289 and 243 amino acids, respectively. The proteins differ internally by 46 amino acids that are unique to the 13S protein. The inventors previous studies have demonstrated that both the 12S and 13S E1A gene products are capable of suppressing neu gene expression (see U.S. Pat. Nos. 5,814,315, 5,651,964, 5,641,484, and 5,643,567, the entire texts of each being specifically incorporated herein by reference). In the practice of the present invention, it is proposed, that one may employ either product interchangeably, or both together.

In other embodiments, numerous other E1A protein species, that are generated as a result of extensive postranslational modification of the primary translation products and have been seen by PAGE analysis (Harlow et al., 1985), are contemplated as useful.

In yet other embodiments, a mini-E1A gene product, described in U.S. Pat. No. 6,197,754, the text of which is completely incorporated herein by reference; and also in pending U.S. patent application Ser. No. 08/809,021, filed Mar. 19, 1998, also incorporated herein by reference, may also be used. The mini-gene comprises the C-terminal region of the E1A protein which comprises about 80 amino acid residues of the C-terminal which correspond to about amino acids 209-289 of 13S E1A . This relatively small C-terminus region was found to exhibit significant tumor suppression activity in vivo. Additionally, smaller fragments within the 209-289 portion of the 13S E1A gene products, that retain the ability to suppress tumorigenicity, derived by removing or altering residues within this domain, may also be used.

Thus, the mini-E1A gene products that may be used to treat a cancer can comprises a C-terminal segment of E1A located within exon 2 of E1A . The mini-E1A gene product may lack a portion of the N-terminal segment. It is contemplated that the mini-E1A gene may lack at least a 10, 15, 20, 25, or 30 amino acid portion of the N-terminal segment of E1A .

Some other embodiments involve an E1A protein, peptide, or polypeptide or nucleic acid encoding the E1A protein, peptide, or polypeptide from which the CR1 region has been removed. Alternatively, E1A gene product can be an E1A gene product from which the CR2 region has been removed. Yet alternatively, the E1A gene product can be an E1A protein, peptide, or polypeptide or nucleic acid encoding the E1A protein, peptide, or polypeptide from which both the CR1 and CR2 regions have been removed.

The E1A gene product may also comprises an amino-terminal segment having the amino acid sequence found between about amino acid 4 and about amino acid 25 of an E1A gene product. The E1A gene product may also comprises an amino-terminal segment having the amino acid sequence found between about amino acid 40 and about amino acid 80 of an E1A gene product.

The E1A protein, peptide, or polypeptide can further comprises a spacer at the C-terminal end of the CR1 domain of an E1A protein, peptide, or polypeptide or nucleic acid encoding the E1A protein, peptide, or polypeptide. The spacer can comprises an amino acid segment having the amino acid sequence found between about amino acid 81 and about amino acid 101 of an E1A protein, peptide, or polypeptide or nucleic acid encoding the E1A protein, peptide, or polypeptide.

From this discussion it will be clear that one may use either the entire E1A gene encoding the E1A protein, or protein; or any portion of the E1A gene or its protein, that has the suitable tumor suppressive properties for the therapy of Akt and/or p38 associated cancers.

B. In vivo Delivery and Treatment Protocols.

Where a gene itself is employed to introduce gene products, a convenient method of introduction will be through the use of a recombinant vector which incorporates the desired gene, generally together with associated control sequences that promote and/or regulate expression of the gene. The preparation of such recombinant vectors as well as the use of various control sequences is well known to those of skill in the art and described in many references, such as, for example, Sambrook et al. (1989), specifically incorporated herein by reference.

In vectors, it is understood that the DNA coding sequences to be expressed (in this case those encoding the oncogenesis-suppressing gene products) are generally positioned adjacent to and under the control of a promoter. It is understood in the art that to bring a coding sequence under the control of such a promoter, one generally positions the 5′ end of the transcription initiation site of the transcriptional reading frame of the gene product to be expressed “downstream” of (i.e., 3′ of) the chosen promoter. As is also known in the art, altering the spacing between a promoter and a nearby transcriptional start site can often be used to influence of the level of expression of the transcript. Where a heterologous promoter is to be used to drive expression of a gene (i.e., a non-E1A promoter in the case of an E1A gene), one can initially employ a spacing that is similar to that between the heterologous promoter and the gene it normally controls (typically less than 100 to several hundred base pairs). However, optimization of expression can involve moving the promoter closer or further from the transcriptional start site. One may also desire to incorporate into the transcriptional unit of the vector an appropriate polyadenylation site (e.g., 5′-AATAAA-3′ ), if one was not contained within the original inserted DNA. Typically, these poly A addition sites are placed about 30 to 2000 nucleotides “downstream” of the coding sequence at a position prior to transcription termination.

While the control sequences of the specific gene (i.e., the E1A promoter) can be employed, other control sequences that function in the cell can also be used. A variety of such promoters, including both constitutive promoters and inducible promoters have been described in the art and are generally available from numerous sources including, e.g., ATCC and commercial sources. Thus, one may mention other useful promoters by way of example, including, e.g., an SV40 early promoter, a CMV promoter, a long terminal repeat promoter from retrovirus, an actin promoter, a heat shock promoter, a metallothionein promoter, and the like.

For introduction of the E1A gene, one will generally desire to employ a vector construct that will also facilitate delivery of the desired gene to the affected cells which may require that the construct be delivered to the targeted tumor cells (e.g., breast, ovarian, pancreatic, colon, prostate, or other tumor cells) in a patient. One way of facilitating delivery is by the use of a viral vector to carry the E1A sequences to efficiently infect a tumor, or pre-tumorous tissue. Exemplary viral vectors include adenoviral, retroviral, vaccinia viral, and adeno-associated viral vectors. These and/or other viral vectors have been successfully used to deliver desired sequences to cells with high infection efficiency.

Commonly used viral promoters for expression vectors are derived from polyoma, cytomegalovirus, Adenovirus, and Simian Virus 40 (SV40). The early and late promoters of SV40 virus are particularly useful because both are obtained easily from the virus as a fragment which also contains the SV40 viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism.

An exemplary vector, that might be used as a starting point for construction, is the E1A containing retroviral vector, termed pSVXE1A-G, described by Robert et al., 1985. This vector comprises the E1A gene which has been brought under the control of the SV-40 early promoter. The inventors propose that this or an E1A construct could either be used directly in the practice of the invention, or could be used as a starting point for the introduction of other more desirable promoters such as those discussed above.

(i) Adenovirus. One method for in vivo delivery of tumor-suppressing gene of the present invention involves the use of an adenovirus vector. An “adenovirus expression vector” is meant to include those constructs containing adenovirus sequences sufficient to (a) support packaging of the construct and (b) to express a polynucleotide that has been cloned therein. Adenoviral transfer of E1A is especially convenient, because E1A is itself an adenoviral gene. Therefore, there need be no non-viral genetic sequences inserted into an adenoviral vector to accomplish adenoviral delivery of E1A .

An exemplary expression vector comprises a genetically engineered form of adenovirus. Knowledge of the genetic organization of adenovirus, a 36 kb, linear double-stranded DNA virus, allows substitution of large pieces of adenoviral DNA with foreign sequences up to 7 kb (Grunhaus and Horwitz, 1992). In contrast to retrovirus, the infection of adenoviral DNA in host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells and can infect a number of other cells as well.

Adenovirus is a particularly suitable gene transfer vector because of its midsized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and some cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, located at 16.8 mμ is particularly efficient during the late phase of infection, and all the mRNAs issued from this promoter possess a 5′-tripartite leader (TL) sequence which makes them preferred mRNAs for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure. Use of the YAC system is an alternative approach for the production of recombinant adenovirus.

A preferred method of introducing the E1A gene to an animal is to introduce a replication-deficient adenovirus containing the E1A gene. An example of such an adenovirus is Ad.E1A (+). Since adenovirus is a common virus infecting humans in nature and the E1A gene is part of a gene that is present in native adenovirus, the use of a replication deficient E1A virus to introduce the gene can effect efficient delivery and expression of E1A protein, peptide, or polypeptide or nucleic acid encoding the E1A protein, peptide, or polypeptides in target cells.

The replication-deficient E1A virus made for example by E1B and E3 deletion also avoids the viral reproduction inside the cell and transfer to other cells, which means the viral infection activity is effectively limited to the first infected target cell. The E1A protein, peptide, or polypeptide or nucleic acid encoding the E1A protein, peptide, or polypeptide(s) is still expressed inside such cells. Also, unlike retrovirus, which can only infect proliferating cells, adenovirus is able to transfer the E1A gene into both proliferating and non-proliferating cells possibly by stimulating non-proliferating cells. Further, the extrachromosomal location of adenovirus in the infected cells decreases the chance of cellular oncogene activation within the treated animal.

While the replication-competent adenovirus may thus be used directly to transfer the E1A gene into cancer cells, replication-competent virus can produce large amounts of adenovirus in the human body and therefore might cause potential side effects due to the replication-competent nature of the wild type adenovirus. It is therefore an advantage to use the replication-deficient adenovirus such as E1B and E3 deletion mutant Ad.E1A (+) to prevent such side effects. In fact, many modifications in the native adenovirus will result in a modified virus that will be useful for the purpose of the invention. Further modification of adenovirus such as E2A deletion may improve the E1A product expression efficiency and reduce the side effects. The only requirement of a native or modified adenovirus is that it should be able to deliver a E1A gene that can be expressed in a target cell in order to have the utility of the invention.

Introduction of adenovirus containing the tumor-suppressing gene (e.g. a E1A gene) into a suitable host is typically done by injecting the virus contained in a buffer. As discussed above, it is advantageous if the adenovirus vector is replication defective, or at least conditionally defective. The adenovirus may be of any of the 42 different known serotypes or subgroups A-F. Adenovirus type 5 of subgroup C is presently preferred starting material for obtaining conditional replication-defective adenovirus vector for use in the present invention. This is because Adenovirus type 5 is a human adenovirus about which the most biochemical and genetic information is known, and it has historically been used for most constructions employing adenovirus as a vector.

Where a E1A gene is used in an adenovirus vector, it can occupy the position normally occupied by the E1A gene, or it can be placed at another position in an adenovirus construct.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. No side effects have been reported in studies of vaccination with wild-type adenovirus (Couch et al., 1963; Top et al., 1971), demonstrating their general safety and therapeutic potential as in vivo gene transfer vectors.

Adenoviruses have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

(ii) Retroviruses. The retroviruses are a group of single-stranded RNA viruses characterized by an ability to convert their RNA to double-stranded DNA to infected cells by a process of reverse-transcription (Coffin, 1990). The resulting DNA can stably integrate into cellular chromosomes as a provirus and direct synthesis of viral proteins. The integration results in the retention of the viral gene sequences in the recipient cell and its descendants. The retroviral genome contains three genes, gag, pol, and env, that code for capsid proteins, polymerase enzyme, and envelope components, respectively. A sequence found upstream from the gag gene, termed PSI components is constructed (Mann et al., 1983). When a recombinant plasmid containing a human cDNA, together with the retroviral LTR and PSI sequences is introduced into this cell line (by calcium phosphate precipitation for example), the PSI sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vector particles are able to infect a broad variety of cell types. However, integration and stable expression require the division of host cells (Paskind et al., 1975).

A novel approach designed to allow specific targeting of retrovirus vectors was developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989). There are certain potential limitations to the use of retrovirus vectors. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990). One limitation to the use of retrovirus vectors in vivo is the limited ability to produce retroviral vector titers greater than 10⁶ infectious U/mL. Titers 10- to 1,000-fold higher are necessary for many in vivo applications.

More recent approaches to the use of retroviral vectors for directing the delivery of genes to particular target cells, such as cancer cells, which would avoid many of these limitations have been described by Paul and Overell (Targeted Genetics Corporation) in U.S. Pat. No. 5,736,387.

(iii) Lentiviral Vectors. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene, such as a E1A gene of this invention, into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the E1A protein, peptide or polypeptide, encoding polynucleotide sequence herein, is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate etc. and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Lentiviral transfer vectors Naldini et al., (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., (1997). Thus, in the present invention, transfect cells with the recombinant lentivirus expressing an E1A protein, peptide, or polypeptide, encoding nucleic acid, ex vivo, or directly infect cells in vivo.

(iv) Other Viral Vectors as Expression Constructs. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and hepadnaviruses may be employed. They offer several attractive features for various mammalian cells (Friedmann, 1989; Ridgeway, 1988; Baichwal and Sugden, 1986; Coupar et al., 1988; Horwich et al., 1990).

A variety of advantages associated with the use of AAV vectors for gene delivery, and methods and compositions for the preparation of such vectors, have been described by Targeted Genetics Corporation and collaborators; see, e.g., Allen et al., WO96/17947; Flotte et al., U.S. Pat. No. 5,658,776. Additional references describing AAV vectors which could be used in the methods of the present invention include the following: Carter, B., 1990; Carter, 1992; Muzyczka, 1992; Flotte, 1992; Chatterjee et al., 1995; Kotin, 1994; Flotte, 1995; and Du et al., 1996.

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Cultures media containing high titers of the recombinant virus were used to infect primary duckling hepatocytes. Stable CAT gene expression was detected for at least 24 days after transfection (Chang et al., 1991).

(v) Lipid-Based Gene Delivery. In a further embodiment of the invention, the expression construct may be associated with one or more lipids. As is known in the art of lipid-based gene delivery, such nucleic acid-lipid complexes can be in a variety of different forms depending generally on the nature of the lipid employed, the ratio of nucleic acid to lipid and/or other possible components, and the method by which the complex is formed. Exemplary types of complexes include structured complexes such as liposomes and micelles, as well as relatively unstructured complexes such as lipid dispersions. By way of illustration, liposomes are vesicular structures generally characterized by a bilayer membrane, such a phospholipid bilayer, enclosing an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes. The present invention thus also provides particularly useful methods for introducing E1A protein, peptide, or polypeptide or nucleic acid encoding the E1A protein, peptide, or polypeptides into cells. One method of in vivo gene transfer which can lead to expression of genes transfected into cells involves the use of liposomes. Liposomes can be used for both in vitro and in vivo transfection. Liposome-mediated gene transfer seems to have great potential for certain in vivo applications in animals (Nicolau et al., 1987). Studies have shown that intravenously injected liposomes are taken up essentially in the liver and the spleen, by the macrophages of the reticuloendothelial system. The specific cellular sites of uptake of injected liposomes appears to be mainly spleen macrophages and liver Kupffer cells. Intravenous injection of liposome/DNA complexes can lead to the uptake of DNA by these cellular sites, and result in the expression of a gene product encoded in the DNA (Nicolau, 1983). The inventors contemplate that E1A protein, peptide, or polypeptide or nucleic acid encoding the E1A protein, peptide, or polypeptides can be introduced into cells using liposome-mediated gene transfer. It is proposed that such constructs can be coupled with liposomes and directly introduced via a catheter, as described by Nabel et al. (1990). By employing these methods, the tumor-suppressing gene products of the present invention can be expressed efficiently at a specific site in vivo, not just the liver and spleen cells which are accessible via intravenous injection. Therefore, this invention also encompasses compositions of DNA constructs encoding E1A protein, peptide, or polypeptide or nucleic acid encoding the E1A protein, peptide, or polypeptide formulated as a DNA/liposome complex and methods of using such constructs.

Liposomes, micelles, and lipid dispersions can be prepared using any of a variety of lipid components (and potentially other components) that can be complexed with nucleic acid or which can entrap e.g., an aqueous compartment comprising a nucleic acid. Illustrative molecules that can be employed include phosphatidylcholine (PC), phosphatidylserine (PS), cholesterol (Chol), N-[1-(2,3-dioleyloxy)propyl]-N,N trimethylammonium chloride (DOTMA), dioleoylphosphatidylethanolamine (DOPE), and/or 3β[N-(N′,N′-dimethylamino-ethane)-carbarmoyl cholesterol (DC-Chol), as well as other lipids known to those of skill in the art. Those of skill in the art will recognize that there are a variety of lipid-based transfection techniques which will be useful in the present invention. Among these techniques are those described in Nicolau et al., 1987, Nabel et al., 1990, and Gao et al., 1991. The inventors have had particular success with lipid/DNA complexes comprising DC-Chol. More particularly, the inventors have had success with lipid/DNA complexes comprising DC-Chol and DOPE which have been prepared following the teachings of L. Huang and collaborators (see, e.g. Gao et al., 1991; Epand et al., PCT/US92/07290, and U.S. Pat. No. 5,283,185). Lipid complexes comprising DOTMA, such as those which are available commercially under the trademark Lipofectin™, from Vical Inc., San Diego, Calif., may also be used. A variety of improved techniques for lipid-based gene delivery that can be employed to deliver genes such as those disclosed herein have been described by L. Huang and collaborators (Deshmukh et al., PCT/US97/06066; Liu et al., PCT/US96/15388, and Huang et al., PCT/US97/12544).

Lipid/nucleic acid complexes can be introduced into contact with cells to be transfected by a variety of methods. In cell culture, the complexes can simply be dispersed in the cell culture solution. For application in vivo, the complexes are typically injected. Intravenous injection allows lipid-mediated transfer of complexed DNA to, for example, the liver and the spleen. In order to allow transfection of DNA into cells which are not accessible through intravenous injection, it is possible to directly inject the lipid-DNA complexes into a specific location in an animal's body. For example, Nabel et al. teach injection of liposomes via a catheter into the arterial wall. In another example, the present inventors have used intraperitoneal injection of lipid/DNA complexes to allow for gene transfer into mice.

The present invention also contemplates compositions comprising a lipid complex. This lipid complex will generally comprise a lipid component and a DNA segment encoding a tumor-suppressing gene. The tumor-suppressing gene employed in the lipid complex can be, for example, an E1A gene. An E1A gene may be similarly complexed with a lipid to form a lipid/DNA complex for gene delivery.

The lipid employed to make the lipid complex can be any of the above-discussed lipids. In particular, DOTMA, DOPE, and/or DC-Chol may form all or part of the lipid complex. The inventors have had particular success with complexes comprising DC-Chol. In a preferred embodiment, the lipid complex comprises DC-Chol and DOPE. While many ratios of DC-Chol to DOPE can have utility, it is anticipated that those comprising a ratio of DC-Chol:DOPE between 1:20 and 20:1 will be particularly advantageous. The inventors have found that lipid complexes prepared from a ratio of DC-Chol:DOPE of about 1:10 to about 1:5 have been particularly useful from the standpoint of stability as well as efficacy. Lipid and liposomes that may be used in conjunction with delivery of E1A protein, peptide, or polypeptide or nucleic acid encoding the E1A protein, peptide, or polypeptides are also described in U.S. Pat. Nos. 5,922,688, 5,814,315, 5,651,964, 5,641,484, and 5,643,567, the entire texts of each being specifically incorporated herein by reference; also see pending U.S. patent application Ser. No. 08/809,021, filed Mar. 19, 1998, also incorporated herein by reference.

In certain embodiments of the invention, the lipid may also be complexed with a hemagglutinating virus (HVJ). This has been shown to facilitate fusion with the cell membrane and to promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. Work by Huang and collaborators has also provided a number of lipid-based gene delivery compositions, some comprising nucleic acid condensing agents and other components; and has further described detailed techniques that can be used for the production of such gene delivery complexes (see, e.g., Targeted Genetics Corporation PCT/US97/12544 as well as other references by Huang et al. above).

In that such expression constructs have been successfully employed in transfer and expression of nucleic acid in vitro and in vivo, then they are applicable for the present invention. As is known in the art, one can also include other components within the gene delivery complex, including proteins and/or other molecules that facilitate targeting to particular cells, binding and uptake by targeted cells, localization within particular subcellular compartments (e.g., the nucleus or cytosol), as well as integration and/or expression of the DNA delivered. A variety of such individual components, and combinations thereof, have been described by Targeted Genetics Corporation in PCT/US95104738.

(vi) Other Non-Viral Vectors. In order to effect expression of sense or antisense gene constructs, the expression construct must generally be delivered into a cell. This delivery may be accomplished in vitro, as in laboratory procedures for transforming cells lines, or in vivo or ex vivo (see below), as in the treatment of certain disease states. As described above, delivery may be via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Improved approaches to non-viral gene delivery using gene delivery fusion proteins (GDFPs) have been described by Overell and Weisser (Targeted Genetics Corporation) in PCT/US95/04738.

Once the expression construct has been delivered into the cell the nucleic acid encoding the gene of interest may be positioned and expressed at different sites. In certain embodiments, the nucleic acid encoding the gene may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of CaPO₄ precipitates into liver and spleen of adult and newborn mice demonstrating active viral replication and acute infection. Benvenisty et al., (1986) also demonstrated that direct intraperitoneal injection of CaPO₄ precipitated plasmids results in expression of the transfected genes. It is envisioned that DNA encoding a gene of interest (e.g., a E1A gene) may also be transferred in a similar manner in vivo and express the gene product.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force. The microprojectiles used have consisted of biologically inert substances such as tungsten or gold beads.

Selected organs including the liver, skin, and muscle tissue of rats and mice have been bombarded in vivo (Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. By virtue of cell type-specific distribution of various receptors, the delivery can be made highly specific (Wu and Wu, 1987; 1988; Overell and Weisser (Targeted Genetics Corporation) in PCT/US95/04738).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a lipid complex. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without lipids. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells, in vitro, and then the return of the modified cells back into an animal. This may involve the surgical removal of tissue/organs from an animal or the primary culture of cells and tissues. See, e.g., Anderson et al., U.S. Pat. No. 5,399,346.

C. Pharmaceutical Compositions and Routes of Administration

Compositions of the present invention will have an effective amount of a gene for therapeutic administration, optionally in combination with an effective amount of a second agent, for example a chemotherapeutic agent or any other anti-cancer agent as exemplified above. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, rinses, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by, e.g., orthotopic, intradermal, subcutaneous, intramuscular, intraperitoneal or intravenous injection. Such compositions would normally be administered as pharmaceutically acceptable compositions, described supra.

The vectors of the present invention are advantageously administered in the form of injectable compositions either as liquid solutions or suspensions. Solid forms suitable for solution in, or suspension in, liquid prior to injection also may be prepared. These preparations also may be emulsified. A typical composition for such purposes comprises 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions can take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All the essential materials and reagents required for inhibiting tumor cell proliferation may be assembled together in a kit. When the components of the kit are provided in one or more liquid solutions, the liquid solution preferably is an aqueous solution, with a sterile aqueous solution being particularly preferred.

For in vivo use, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

Parenteral Administration. The active compounds of the present invention will often be formulated for parenteral administration, e.g., formulated for injection via the intravenous, intramuscular, sub-cutaneous, or even intraperitoneal routes. The preparation of an aqueous composition that optionally contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained. for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of cancerous tissues may be accomplished in any one of a variety of ways. Plasmid vectors and retroviral vectors, adenovirus vectors, and other viral and non-viral vectors all present means by which to target human cancers. The inventors anticipate particular success for the use of lipid-based complexes to target E1A genes to cancer cells. Intravenous injection can be used to direct genes to cells, including transformed cells. Directly injecting the complex into the proximity of a cancer can also provide for targeting of the complex with some forms of cancer. For example, cancers of the ovary can be targeted by injecting the lipid mixture directly into the peritoneal cavity of patients with ovarian cancer. Of course, lipid complexes that are selectively taken up by a population of cancerous cells can also be used as described for preferentially targeting the tumor suppressing gene(s) to particular target cells.

Those of skill in the art will recognize that the optimal treatment regimens for using E1A to suppress cancers can be straightforwardly determined. This is not a question of experimentation, but rather one of optimization, which is routinely conducted in the medical arts. The in vivo studies in animal models as described herein provide a starting point from which to begin to optimize the dosage and delivery regimes. The frequency of injection will initially be once a week, as was done in the mice studies. However, this frequency might be optimally adjusted from one day to every two weeks to monthly, depending upon the results obtained from the initial clinical trials and the needs of a particular patient. Human dosage amounts can initially be determined by extrapolating from the amount of E1A used in mice, approximately 15 μg of vector DNA per 50 g body weight. Based on this, a 50 kg woman might require treatment a dose in the range of about 15 mg of DNA per dose. Of course, this dosage amount may be adjusted upward or downward, as is routinely done in such treatment protocols, depending on the results of the initial clinical trials and the needs of a particular patient.

D. Combination Therapies

In order to increase the effectiveness of the combination therapy with the E1A-based gene therapy as described in the present invention, it may be desirable to combine these compositions with yet other agents effective in the treatment of cancer.

In the context of the present invention, it is therefore contemplated that the E1A-based gene therapy will be used in combination with other anticancer-therapies known in the art for treating cancers involving Akt activation and/or p38 inactivation. A variety of cancers involving Akt and p38, including pre-cancers, tumors, malignant cancers can be treated according to the methods of the present invention. Some of the cancer types contemplated for treatment in the present invention include breast, prostate, liver, myelomas, bladder, blood, bone, bone marrow, brain, colon, esophagus, gastrointestine, head, kidney, lung, nasopharynx, neck, ovary, skin, stomach, and uterus cancers.

The administration of the other anti-cancer therapy may precede or follow the E1A-based gene therapy by intervals ranging from minutes to days to weeks. In embodiments where the other anti-cancer therapy and the E1A-based gene therapy are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the other anti-cancer therapy and the E1A-based gene therapy will be required to achieve complete cancer cure. Various combinations may be employed, where the other anti-cancer therapy agent is “A” and the E1A-based gene therapy is “B”, as exemplified below:

```
    A/B/A B/A/B B/B/A A/A/B B/A/A A/B/B B/B/B/A B/B/A/B
    A/A/B/B A/B/A/B A/B/B/A B/B/A/A B/A/B/A B/A/A/B B/B/B/A
    A/A/A/B B/A/A/A A/B/A/A A/A/B/A A/B/B/B B/A/B/B B/B/A/B
```

Other combinations also are contemplated. The exact dosages and regimens of each agent can be suitable altered by those of ordinary skill in the art.

Some examples of other anti-cancer therapies that will be used are described below.

a) Chemotherapeutic Agents

Agents that damage DNA are chemotherapeutics. These can be, for example, agents that directly cross-link DNA, agents that intercalate into DNA, and agents that lead to chromosomal and mitotic aberrations by affecting nucleic acid synthesis. Agents that directly cross-link nucleic acids, specifically DNA, are envisaged and are exemplified by cisplatin, and other DNA alkylating agents. Agents that damage DNA also include compounds that interfere with DNA replication, mitosis, and chromosomal segregation.

Some examples of chemotherapeutic agents include antibiotic chemotherapeutics such as, Doxorubicin, Daunorubicin, Mitomycin (also known as mutamycin and/or mitomycin-C), Actinomycin D (Dactinomycin), Bleomycin, Plicomycin; plant alkaloids such as Taxol, Vincristine, Vinblastine; alkylating agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine; and miscellaneous agents such as, Cisplatin (CDDP), Carboplatin, Procarbazine, Mechlorethamine, Camptothecin, Ifosfamide, Nitrosurea, Etoposide (VP16), Tamoxifen, Tumor Necrosis Factor, Raloxifene, Estrogen Receptor Binding Agents, Gemcitabine, Navelbine, Farnesyl-transferase inhibitors, Transplatinum, 5-Fluorouracil, and Methotrexate, Temazolomide (an aqueous form of DTIC), or any analog or derivative variant of the foregoing.

(i) Antibiotics

Doxorubicin. Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro -6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

Daunorubicin. Daunorubicin hydrochloride, 5,12-Naphthacenedione, (8S-cis)-8-acetyl- 10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course these dosages are exemplary, and any dosage in-between these points is also expected to be of use in the invention.

Mitomycin. Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg, 20 mg, or 10 mg I.V., the maximal serum concentrations were 2.4 mg/mL, 1.7 mg/,mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways.

Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

Actinomycin D. Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors which fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

Bleomycin. Bleomycin is a mixture of cytotoxic glycopeptide antibiotics isolated from a strain of Streptomyces verticillus. It is freely soluble in water. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted. Bleomycin may be given by the intramuscular, intravenous, or subcutaneous routes.

(ii) Miscellaneous Agents

Cisplatin. Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg//m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

Cisplatin may also be used in combination with emodin or emodin-like compounds in the treatment of non-small cell lung carcinoma. Combination of cisplatin and emodin and or emodin-like compounds may also be used for the treatment of any neu-mediated cancers.

VP16. VP16 is also know as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/ m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

Tumor Necrosis Factor. Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

(iii) Plant Alkaloids

Taxol. Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

Vincristine. Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisolone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisolone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisolone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can also be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

Vinblastine. When cells are incubated with vinblastine, dissolution of the microtubules occurs. Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours.

Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine for use will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

(iv) Alkylating Agents

Carmustine. Carmustine (sterile carmustine) is one of the nitrosoureas used in the treatment of certain neoplastic diseases. It is 1,3bis (2-chloroethyl)-1-nitrosourea. It is lyophilized pale yellow flakes or congealed mass with a molecular weight of about 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisolone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m², 50 mg/m², 60 mg/m², 70 mg/m², 80 mg/m², 90 mg/m², 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any e vent, determine the appropriate dose for the individual subject

Melphalan. Melphalan also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject.

Cyclophosphamide. Cyclophosphamide is 2H -1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N-POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

Chlorambucil. Chlorambucil (also known as leukeran) was first synthesized in 1953. It is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remingtons Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

Busulfan. Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

Lomustine. Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours.

The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m², 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

b) Radiotherapeutic agents

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

c) Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

d) Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Immunotherapy could be used as part of a combined therapy, in conjunction with the E1A-based gene therapy-based therapy.

The general approach for combined therapy is discussed below. In one aspect the immunotherapy can be used to target a tumor cell. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and pl55. Alternate immune stimulating molecules also exist including: cytokines such as IL-2, IL -4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with the E1A-based gene therapy based combination therapy of this invention will enhance anti-tumor effects.

(i) Passive Immunotherapy. A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

(ii) Active Immunotherapy. In active immunotherapy, an antigenic peptide, polypeptide or protein, or an autologous or allogenic tumor cell composition or “vaccine” is administered, generally with a distinct bacterial adjuvant (Ravindranath & Morton, 1991).

(iii) Adoptive Immunotherapy. In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro.

e) Gene therapy

In yet another embodiment, other gene therapies in conjunction with the E1A-based gene therapy described in the invention are contemplated. Appropriate genes for use according to this embodiment are genes encoding tumor suppressors (p53, Rb, p16), antisense oncogenes (ras, myc, erb), inducers of apoptosis (Bcl-2, Bax), kinases, and other such genes.

f) Other agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen and this often reduces the risk of metastases.

E. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials & Methods

### Cell Lines and Cultures

The human breast cancer MDA-MB-231 and MCF-7 cells were grown in Dulbecco's modified Eagle's medium/F-12 (Life Technologies, Inc.) supplemented with 10% fetal bovine serum. The E1A stable expressing cell lines were established by transfecting the MDA-MB-231 and MCF-7 cells with pSV-E1A-neo plasmid DNA, as a transfection control, pSV-neo vector were also transfected into these cells. Stable cell lines were isolated by G418 selection (750 μg/ml) and verified by western blot analysis.

### [3]Thymidine Incorporation Assay

The assay was carried out as described previously. Briefly, the cells were trypsinized, plated in 96-well plates (3×10³ cells/well), and incubated for 5 h with 0.5 μCi of [³H]thymidine per well. Labeled medium was then removed and washed with PBS. Cells were released from the plate with 50 ul 1% trypsin/PBS per well and lysed with the automated cell harvester and absorbed the DNA onto glass fiber filters. Dry the filters and seal into a sample tube with 10 ml scintillant. The radioactivity of present in the filters was determined by scintillation counting in the Betaplate liquid scintillation counter.

### MTT Assay

The indirect measure of the viable cells after exposure to Taxol using the metabolic conversion of tetrazolium salt (MTT) to formazan was used. Cells (3×10³/well) were seeded in triplicate in 96-well culture plates in 0.2 ml of culture medium and allowed to adhere for 24 h. After exposure to Taxol for a certain period of time, 20 μl of MTT was then added to each well. Cells were cultured for an additional 2 h, and followed by addition of 100 μl of extraction buffer (20% SDS in 50% N,N-dimethyl formamide, pH 4.7). The cells were incubated overnight at 37° C., and the plates were absorbance at 570 nm was measured.

### Preparation of Total-Cell Lysates, Western Blotting Analysis and Antibodies

Cells were washed once with phosphate-buffered saline and lysed in a lysis buffer containing 20 mM Tris-HCl (p7.5), 150 mM NaCl, 5 mM EDTA, 10 mM NaF, 1% Nonidet P-40, 1 mM PMSF (phenylmethylsulfonyl fluoride, 1 nM NaVO₃ (sodium orthovanadate), and 1.5% aprotinin. The cell extracts were clarified by centrifugation, protein concentration were determined by Bio-Rad protein assay reagent and analyzed in a spectrophotometer using BSA (bovine serum album, Sigma, St. Louis, Wash.) as the protein standards, and aliquots of twenty-five microgram of proteins were resolved by sodium dodecyl sulfate-polyacrylamide gel electrophoresis (SDS-PEGE) and transferred to nitrocellulose membranes (Millipore Corp., Bedford, Mass.) using standard procedures. The membranes were then subjected to Western blot, and the blots were developed with the enhanced chemiluminescence (ECL) system (Amersham).

Immunobloting was performed with the following antibodies: A monoclonal antibody against the E1A proteins, M58 (PharMingen), was used for screening the expression of E1A proteins in stable cell clones. A rabbit polyclonal anti-Bax antibody and hamster anti-human Bcl-2 monoclonal antibody were purchased from Santa Cruz Biotechnology, Inc. (Santa Cruz, Calif.). Rabbit polyclonal antibodies against phospho-specific p38 (p38-p), phospho-specific Akt (Akt-p), and total p38 or Akt were purchased from NEB. In order to detect HA-tagged p38 and Akt, a monoclonal anti-HA antibody was used (Boerhinger &. Mannheim).

### Transient Transfection

Expression vectors for HA-tagged p38MAPK (pMT2T, HA-p38), HA-myr-Akt (constiutively activated Akt, CA-Akt) or HA tagged dominant negative Akt (DN-Akt), and cytomegalovirus (CMV) promoter-luciferase expressing vector (pCMV-luc) were used in this study. 1×10⁵ cells in a 60-mm well dish were transfected with 2.2 μg of total DNA using DC-Chol cationic liposome as described previously. After growing for another 48 hours, the cells were split into three sets, one set of cells was used for luciferase assay after exposure with or without Taxol for 24 hours, another one for analysis of the protein expression of Akt and 38, and the third one was sent for FACS analysis.

### Immunoprecipatation

After transcient transfection with HA-tagged p38 and/or CA-Akt, cells were stimulated with 10 μM insulin for 15 minutes and cell extracts were prepared by lysing cells in a buffer containing 50 mM HEPES, pH 7.6, 150 mM NaCl, 10 mM EDTA, 2 mM sodium orthovanadate, 100 mM NaF, 0.5 mM PMSF, 1 mM aprotinin and 1% triton X-100 for 15 min at 4° C. Cell lysates were centrifuged at 14,000 rpm for 30 minutes. The supernatants were transferred to a fresh tube. Proteins were cleared by addition of normal mouse or rabbit IgG and immunoprecipated with anti-p38, anti-Akt, or anti-HA antibodies. Immunoprecipatants were resolved on 10% SDS-PEGE and transferred to nitrocellulose membranes. Akt and p38 were detected by Western blotting. As an immunoprecipate positive control of Akt, the same membrane was also reprobed with anti-IKK-a antibody by Western blot.

### Establishment of Orthotopic Breast Cancer Model

To establish orthotopic breast cancer model in nude mice, human MDA-MB-231 cells were inoculated into the mammary fat pad (m.f.p.) of mouse. Tumor size were monitored and caculated on a weekly basis.

### EXAMPLE 2

### Expression of Akt and p38 in Cancer Cells

Active/overexpressed Akt is represented by its phosphorylated form. On screening, it was found that the levels of activated Akt are enhanced in most cancer cells in comparison to immortalized NIH3T3 non-cancer cells. Some of the representative human cancer cell types screened include breast cancer cells, ovarian cancer cells, prostate cancer cells, and colon cancer cell lines. In contrast, phosphorylated p38 protein, which represents the active form of p38, was undetectable in most of these cancer cell lines. Phosphorylated p38 was however detected in the immortalized non-cancer NIH3T3 cells under the same protein loading conditions. This demonstrates that most cancers have upregulated or activated versions of the Akt oncoprotein and downregulated or inactive forms of the pro-apoptotic factor p38.

### EXAMPLE 3

### E1A Downregulates Akt and Upregulates p38 Phosphorylation in Breast Cancer Cells

Breast cancer cell lines, MDA-MB-231 and MCF-7, were transfected with either vector alone (V) (as a control) or with E1A (E), and were then grown exponentially and harvested for preparation of whole cell lysates. Expression of activated phosphorylated p38 (p38-p) and phosphorylated Akt (Akt-p) were detected by polyclonal anti-phospho-specific antibodies against phospho-p38 and phospho-Akt^{S437} (New England Biolab, NEB), respectively. The total p38 and Akt proteins were also detected using polyclonal antibodies against p38 and Akt from NEB. To verify the expression of E1A in the stable cells, a monoclonal antibody against E1A (M58) was used. The phospho-p38 protein level was under the detectable range in both cell lines transfected with vector alone, however phosopho-p38 level was upregulated with introduction of E1A protein. In contrast, the basal phospho-Akt levels were higher in cells transfected with vector alone than in cells transfected E1A , although the total p38 and Akt protein levels were comparable in the different transfectants.

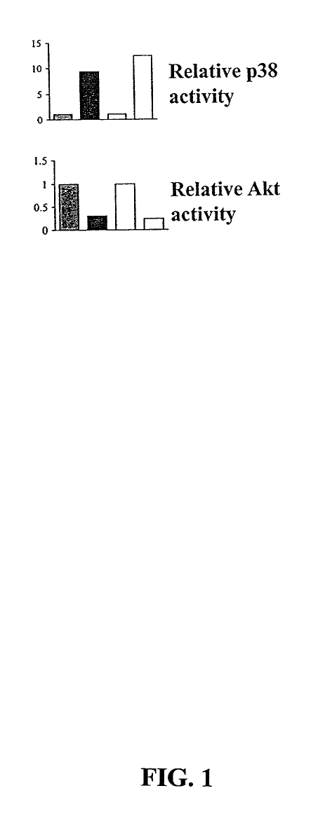

The quantitative difference between the vector and E1A stable cells were compared by converting the band densities by densitometry. The relative p38 activity (p38-p versus p38-c) and relative Akt activity (Akt-p/Akt-c) were plotted separately. These results are illustrated in FIG. 1.

Consistent with the results obtained by using anti-phospho specific antibodies against p38 and Akt, the inventors also found that expressing of E1A in MDA-MB-231 and MCF-7 cells dramatically enhanced p38 kinase activity by using GST-ATF-2 fusion protein as the substrate for p38 kinase. Meanwhile, the Akt kinase activity in these E1A expressing cells were significantly repressed as detected by an Akt-kinase assay using GST-GSK-3-β as the substrate.

The ability of E1A to inhibit the insulin-induced activation of Akt was analyzed. Cells were serum starved overnight and then stimulated with or without 10 μM of insulin for 15 minutes. Cells were then harvested and the Akt phosphorylation (which is the active form of Akt) was detected by using a polyclonal antibody against phospho-specific Akt. The total Akt levels were also detected as protein loading controls. E1A effectively inhibited activation of Akt by insulin.

### EXAMPLE 4

### Akt and p38 are Reciprocally Regulated

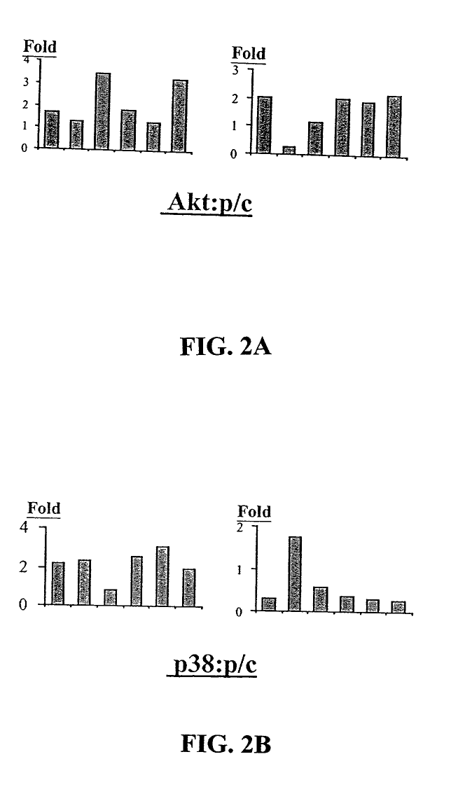

In order to determine the relationships between the Akt pathway and the p38 pathway, experiments were performed with inhibitors of both pathways. Wortmannin was used to inhibit the Akt pathway. Wortmannin is a drug that inhibits P13K which is a signal protein that is directly upstream of Akt in the Akt pathway. A synthetic compound, SB203580, was used as a p38 specific inhibitor. Cells were serum starved overnight and were then treated with either SB203580 (20 μM) or Wortmannin (0.1 μM) for 4 hours, 8, hours, 16 hours, 24 hours, 36 hours. When p38 activity was blocked by SB203580 for 8 hours, the Akt activity was increased by approximately 3 fold in E1A stable expressing MDA-MB-231 cells. Whereas, when Akt activity was blocked by Wortmannin for 4 hours, the p38 activity was enhanced to approximately 6 fold. The relative p38 activity (p38-p versus p38-c) and relative Akt activity (Akt-p versus Akt-c) are illustrated in FIG. 2A and FIG. 2B respectively.

### EXAMPLE 5

### Akt and p38 are not Physically Associated

To detect whether p38 and Akt are physically associated, immunoprecipitation analysis was performed. MDA-MB-231 cells (231) as well 293T cells were co-transfected with HA-tagged constitutively activated Akt and wild type p38. Cells were lysed and harvested after transfection for 36 hours. Cell lysates were incubated with polyclonal antibodies against Akt and p38, respectively. After precipitation by protein-A-agarose beads, the proteins in the beads were washed and dissolved in 30 ml of 2×loading buffer and followed by 10% SDS-PEGE. After transfer to nitrocellulose membrane, the membrane were probed with anti-Akt (1:500) and anti-p38 (1:500) antibodies. Akt and p38 are not physically associated.

### EXAMPLE 6

### Downregulation of Phospho-Akt or Upregualtion of Phospho-p38 is Required for E1A to Inhibit Cell Growth In Vitro

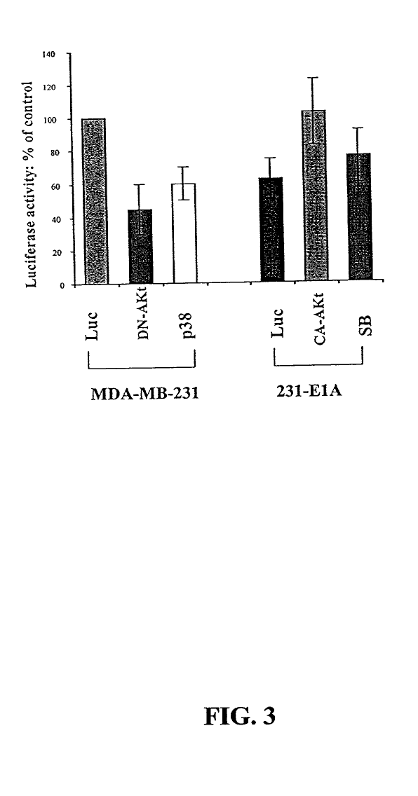

Parental MDA-MB-231 and E1A stable cells were plated into six-well plates in triplicate, after growing for 24 hours. MDA-MB-231 cells were co-transfected with either dominant negative Akt (DN-Akt) or activated p38 plus pcDNA3-Luciferase reporter, while 231-E1A stable cells were co-transfected with either pcDNA3-Luciferase reporter or a constitutively activated Akt (CA-Akt) expressing construct, plus pcDNA3-Luciferase reporter gene. Cells were allow to grow for another 48 hours and then harvested. The luciferase activity was then measured and normalized with protein concentration and these values were compared with the parental MDA-MB-231 cells. The p38 activity was inhibited using SB203580, which was added to the culture medium at a concentration of 20 μM. Cells were grown in the presence of SB203580 for 24 hours before harvesting for luciferase assay. The results of these experiments are shown in FIG. 3, which demonstrates that inactivation of Akt or activation of p38 is required for E1A to repress cell growth.

### EXAMPLE 7

### Repression of Tumorigenicity In Vitro

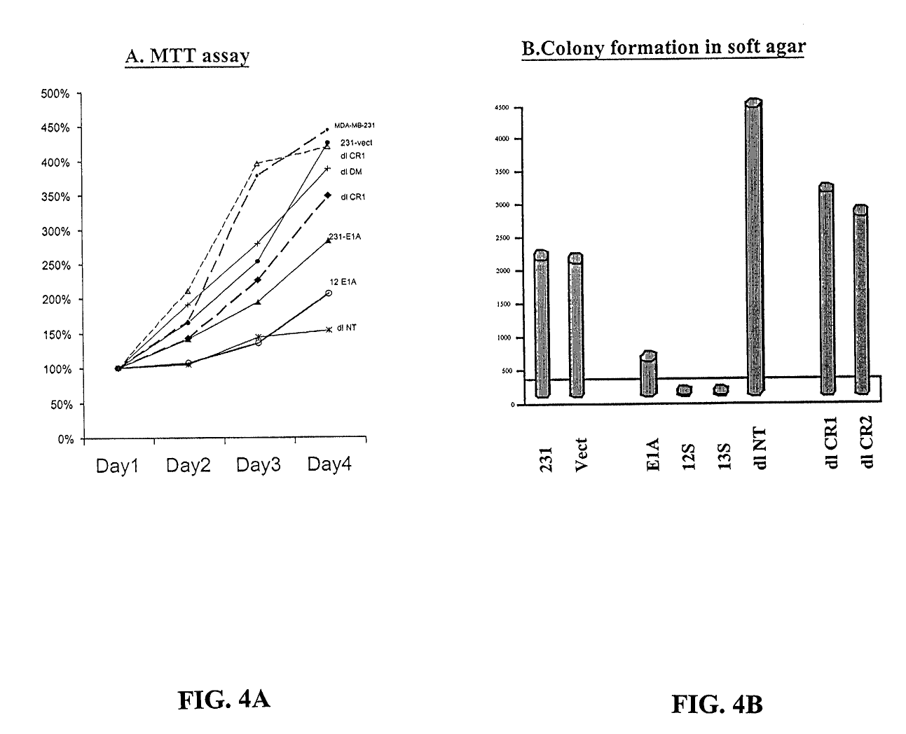

Breast cancer cells (MDA-MB-231) were transfected with either wild type E1A or mutant E1A and cell growth was measured by the MTT assay (see FIG. 4A) as well as a soft agar assay (FIG. 4B). The control comprised the pSV-neo vector alone transfected into cells; WTE1A comprises wild type E1A stable cells; 12S comprises 12S E1A stable cells; dlNT comprises the N-terminal deletion mutation stable cells; dlCR1 comprises the CR1 deletion mutation stable cells; dlCR2 comprises CR2 domain deletion mutation stable cells; and dlDM comprises the N-terminal plus CR2 domain deletion mutation stable cells. Thus, these experiments analyzed the effects of various different E1A protein, peptide, or polypeptide or nucleic acid encoding the E1A protein, peptide, or polypeptides (including E1A mutations) on cancer cells that overexpress Akt. Thus, E1A represses anchorage-dependent and anchorage-independent cells growth in soft agar in Akt-activating breast cancer MDA-MB-231 cells.

### EXAMPLE 8

### Repression of Tumorigenicity In Vivo

An orthotopic breast cancer model was establish in nude mice as described in Example 1 above. Briefly, human MDA-MB-231 cells were inoculated into the mammary fat pad (m.f p.) of mice. For each mouse, two inoculations were performed, one in the upper and the other one in lower mammary fat pad (mfp), five mice were inoculated in each group of the transfectants. For each mfp, 2×10⁶ cells were inoculated. Tumor volume were monitored eight days later after inoculation and followed every 4 days for successive eight weeks. Tumor weight were calculated using the formula:

Tumor weight (gm)=½lw²,

where l represents the length, w stands for width.

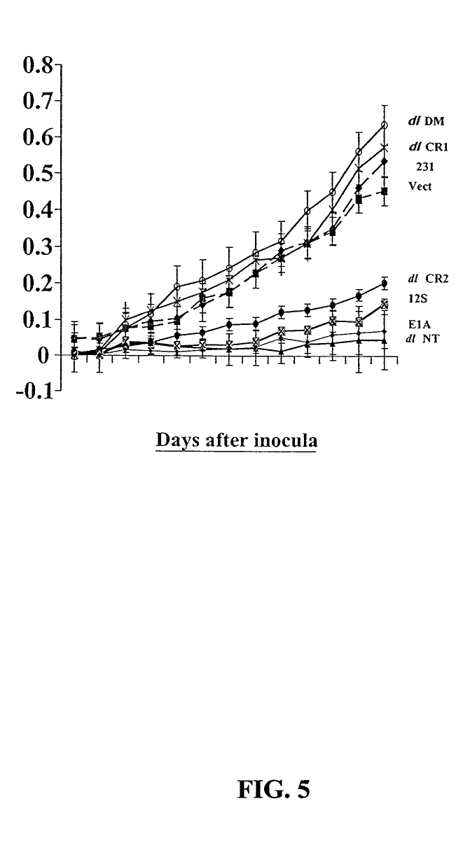

As shown in FIG. 5, wild type E1A as well as 12S E1A dramatically repressed tumor growth in vivo in nude mice whereas, mutation of the CR1 domain or the CR2 plus N-terminal domains abrogated E1A tumor suppressive activity in MDA-MB-231 cells.

### EXAMPLE 9

### Combination of E1A Gene Therapy with Other Chemotherapeutics

Expression of E1A enhanced the anti-tumor effect of the chemotherapeutic drug Taxol in nude mice in vivo and prolonged animal survival rate. Taxol treatment repressed tumor growth both in 231 cells transfected with the vector alone (controls) and 231-E1A stable cell established tumors, however, a more dramatic therapeutic effect was observed in Taxol treatment of 231-E1A cells. TUNEL labeling of apoptotic cells in the tumor tissues with or without treatment of Taxol also confirms this. The treatment groups included the SN vehicle, SN-liposome-E1A, Taxol alone, and Taxol plus SN-E1A. At least 7 animals were included in each group.

It is also contemplated that other chemotherapeutics may be used, such as but not limited to, cisplatin, gemcitabine, novelbine, doxorubicin, VP16, TNF, emodin, daunorubicin, dactinomycin, mitoxantrone, procarbazine, mitomycin, carboplatin, bleomycin, etoposide, teniposide, mechlroethamine, cyclophosphamide, ifosfamide, melphalan, chlorambucil, ifosfamide, melphalan, hexamethylmelamine, thiopeta, busulfan, carmustine, lomustine, semustine, streptozocin, dacarbazine, adriamycin, 5-fluorouracil (5FU), camptothecin, actinomycin-D, hydrogen peroxide, nitrosurea, plicomycin, tamoxifen, transplatinum, vincristin, vinblastin, TRAIL, or methotrexate.

### EXAMPLE 10

### Human Treatment Protocols

Based on the results of the in vivo animal studies described above, those of skill in the art will understand and predict the enormous potential for human treatment of cancers with E1A protein, peptide, or polypeptide or nucleic acid encoding the E1A protein, peptide, or polypeptides that may be complexed to lipids and lioposomes. Indeed, as noted above, success in the animal models has merited the approval and initiation of Phase I human clinical trials which are now ongoing at a number of centers. It is expected that these clinical trials will confirm the use of E1A, and other tumor-suppressing gene products for the treatment of cancers in humans. Dosage and frequency regimes will initially be based on the data obtained from in vivo animal studies, as was described above.

The treatment of human cancers is possible by the introduction of the E1A gene. This may be achieved most preferably by introduction of the desired gene through the use of a viral or non-viral vector to carry the E1A sequences to efficiently infect the tumor, or pre-tumorous tissue. Viral vectors will preferably be an adenoviral, a retroviral, a vaccinia viral vector or adeno-associated virus (Muro-cacho et al., 1992). These vectors are preferred because they have been successfully used to deliver desired sequences to cells and tend to have a high infection efficiency. Hung et al. have conducted studies showing that native adenovirus can be employed to transfer the E1A gene in accordance with the invention. However, a particularly preferred type of adenovirus is the group of replication-deficient adenoviruses.

As noted above, overexpression (or activation) of the human Akt oncogene and/or downregulation (or inactivation) of human p38 is a frequent event in many types of human cancers, including cancers of the breast, ovarian, prostate, pancreatic, colon cancer, etc.

This example describes a protocol to facilitate the treatment of cancer using the E1A protein, peptide, or polypeptide or a nucleic acid encoding the E1A protein, peptide, or polypeptides, in such cancers. A patient presenting a cancer may be treated using the following protocol. Patients may, but need not, have received previous chemo- radio- or gene therapeutic treatments. Optimally the patient will exhibit adequate bone marrow function (defined as peripheral absolute granulocyte count of >2,000/mm3 and platelet count of 100,000/mm3, adequate liver function (bilirubin 1.5 mg/dl) and adequate renal function (creatinine 1.5 mg/dl).

For tumors over-expressing Akt, the levels of Akt expression can be monitored before, during, and after the therapy. Akt expression may be measured by measuring immunological methods. Briefly, sections of 3- to 4 mm thickness of the primary tumors and of the cell block preparations are cut, deparaffinized in xylene, and rehydrated in descending grades (100-70%) of ethanol. Endogenous peroxidase activity is blocked with 3% hydrogen peroxide in methanol. After several washes in distilled water and phosphate-buffered saline, the sections are incubated with a 1:10 dilution of normal horse serum to minimize background staining. This is followed by incubation for 1 hr at room temperature with a primary antibody. The peroxidase staining procedure utilizes ABC Elite Kits (Vector Laboratories, Burlingame, Calif). The immunostaining reactions are visualized using 3-amino-9-ethylcarbazole as the chromogen. The sections and/or cytospin preparations are stained with toluidine blue and mounted in permount. Positive and negative control immunostains are also prepared.

For tumors underexpressing p38, the levels of p38 expression will be monitored before, during, and after the therapy. p38 expression may be measured by measuring immunological methods similar to those described above with an anti-p38 Ab or a anti-phospho p38 Ab.

The sections are reviewed by a pathologist. Two features of the immunoreaction will be recorded using a semi quantitative scale: the relative number of positive cells (0%, <10%, 10-50%, and >50%) and the intensity of the reaction (0-3). The pattern of immunostaining (membranous, cytoplasmic) is recorded separately. A tumor is considered Akt positive and/or p38 negative if any neoplastic cells show positive staining with antibody against phospho-Akt and/or negative staining with antibody against phospho-p38, respectively. A breast carcinoma known for its strong positive membrane staining will be used as a positive control. The quantitative measurement of phospho-Akt and/or phospho-p38 immunostaining can be performed using computerized image analysis with the SAMBA 4000 Cell Image Analysis System (Image Products International, Inc., Chantilly, Va.) integrated with a Windows based software. A strong staining tumor tissue section will be used as positive control. The primary antibody will be replaced by an isotype-matched irrelevant antibody to set the negative control threshold, averaging the results from ten fields.

Protocol for the Treatment of Cancer Using E1A Gene Products. A composition of the present invention is typically administered parenterally or orally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, or infusion techniques. The E1A protein, peptide, or polypeptide or a nucleic acid encoding the E1A protein, peptide, or polypeptides, and/or other tumor suppressing product may be delivered to the patient before, after or concurrently with the other anti-cancer agents. A typical treatment course may comprise about six doses delivered over a 7 to 21 day period. Upon election by the clinician the regimen may be continued six doses every three weeks or on a less frequent (monthly, bimonthly, quarterly etc.) basis. Of course, these are only exemplary times for treatment, and the skilled practitioner will readily recognize that many other time-courses are possible.

A major challenge in clinical oncology is that many tumor cells are resistant to chemotherapeutic treatment. One goal of the inventors' efforts has been to find ways to improve the efficacy of chemotherapy. In the context of the present invention, the E1A protein, peptide, or polypeptide or a nucleic acid encoding the E1A protein, peptide, or polypeptides, can be combined with any of a number of conventional chemotherapeutic regimens.

To kill cancer cells using the methods and compositions described in the present invention, one will generally contact a target cell with the E1A protein, peptide, or polypeptide or a nucleic acid encoding the E1A protein, peptide, or polypeptides, and at least one chemotherapeutic agent (second agent), examples of which are described above. These compositions will be provided in a combined amount effective to kill or inhibit the proliferation of the cell. This process may involve contacting the cell with the E1A protein, peptide, or polypeptide or a nucleic acid encoding the E1A protein, peptide, or polypeptides, and the second agent at the same time. Alternatively, this process may involve contacting the cell with a single composition or pharmacological formulation that includes both agents or by contacting the cell with two distinct compositions or formulations at the same time, wherein one composition includes the E1A protein, peptide, or polypeptide or a nucleic acid encoding the E1A protein, peptide, or polypeptides, and the other includes the second agent.

Alternatively, the E1A protein, peptide, or polypeptide or a nucleic acid encoding the E1A protein, peptide, or polypeptides, administration may precede or follow the delivery of the second agent by intervals ranging from minutes to weeks. In embodiments where the E1A protein, peptide, or polypeptide or a nucleic acid encoding the E1A protein, peptide, or polypeptides, and the second compound are applied separately, one would ensure that a significant period of time did not expire between the time of each delivery, such that the second agent and the E1A protein, peptide, or polypeptide or a nucleic acid encoding the E1A protein, peptide, or polypeptides, would still be able to exert an advantageously combined effect on the cancer. In such instances, it is contemplated that one would contact the cell with both agents within about 6 hours to one week of each other and more preferably, within 24-72 hours of each other. In some situations however, it may be desirable to extend the time period for treatment significantly where several days (2, 3, 4, 5, 6, 7 or more) to several weeks (1, 2, 3, 4, 5, 6, 7 or more) lapse between respective administrations.

Regional delivery of the E1A protein, peptide, or polypeptide or a nucleic acid encoding the E1A protein, peptide, or polypeptides, will be an efficient method for delivering a therapeutically effective dose to counteract the clinical disease. Likewise, the chemotherapy may be directed to a particular effected region. Alternatively systemic delivery of either, or both, agent may be appropriate. The therapeutic composition of the present invention is administered to the patient directly at the site of the tumor. This is in essence a topical treatment of the surface of the cancer. The volume of the composition should usually be sufficient to ensure that the entire surface of the tumor is contacted by the E1A protein, peptide, or polypeptide or a nucleic acid encoding the E1A protein, peptide, or polypeptides, and second agent. In one embodiment, administration simply entails injection of the therapeutic composition into the tumor. In another embodiment, a catheter is inserted into the site of the tumor and the cavity may be continuously perfused for a desired period of time.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

Of course, the above-described treatment regimes may be altered in accordance with the knowledge gained from clinical trials such as those described herein. Those of skill in the art will be able to take the information disclosed in this specification and optimize treatment regimes based on the clinical trials described in the specification.

### EXAMPLE 11

### Clinical Trials

This example is concerned with the development of human treatment protocols using the E1A protein, peptide, or polypeptide or a nucleic acid encoding the E1A protein, peptide, or polypeptides, alone or in combination with other anti-cancer drugs. The E1A protein, peptide, or polypeptide or a nucleic acid encoding the E1A protein, peptide, or polypeptides, and anti-cancer drug treatment will be of use in the clinical treatment of various cancers involving Akt activation or p38 inactivation in which transformed or cancerous cells play a role. Such treatment will be particularly useful tools in anti-tumor therapy, for example, in treating patients with ovarian, breast, prostate, pancreatic, brain, colon, and lung cancers that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the E1A protein, peptide, or polypeptide or a nucleic acid encoding the E1A protein, peptide, or polypeptides, in clinical trials.

Patients with advanced, metastatic breast, epithelial ovarian carcinoma, pancreatic, colon, or other cancers chosen for clinical study will typically have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to the E1A protein, peptide, or polypeptide or a nucleic acid encoding the E1A protein, peptide, or polypeptides, and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, PSA, p38 (phosphorylated and un-phosphorylated forms), Akt (phosphorylated and un-phosphorylated forms) and in the cells (E1A proteins, peptides or polypeptides or nucleic acids encoding the same) may be assessed and recorded.

In the same procedure, the E1A protein, peptide, or polypeptide or a nucleic acid encoding the E1A protein, peptide, or polypeptides, may be administered alone or in combination with the other anti-cancer drug. The administration may be in the pleural/peritoneal cavity, directly into the tumor, or in a systemic manner. The starting dose may be 0.5 mg/kg body weight. Three patients may be treated at each dose level in the absence of grade >3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of the E1A protein, peptide, or polypeptide or a nucleic acid encoding the E1A protein, peptide, or polypeptides, and the lot of anti-cancer drug exceed 5 EU/kg for any given patient.

The E1A protein, peptide, or polypeptide or a nucleic acid encoding the E1A protein, peptide, or polypeptides, and/or the other anti-cancer drug combination, may be administered over a short infusion time or at a steady rate of infusion over a 7 to 21 day period. The E1A protein, peptide, or polypeptide or a nucleic acid encoding the E1A protein, peptide, or polypeptides, infusion may be administered alone or in combination with the anti-cancer drug and/or emodin like tyrosine kinase inhibitor. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the E1A protein, peptide, or polypeptide or a nucleic acid encoding the E1A protein, peptide, or polypeptides, in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored e.g. CEA, CA 15-3, p38 (phosphorylated and non-phopshorylated forms) and Akt (phosphorylated and non-phosphorylated forms), p185, etc.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, p185, p38, Akt) and in the cells (p38, Akt) may be assessed. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. An urinalysis may be performed every 4 weeks.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

U.S. Pat. No. 5,399,346

U.S. Pat. No. 4,394,448

U.S. Pat. No. 4,920,211

U.S. Pat. No. 5,516,631

U.S. Pat. No. 5,578,482

U.S. Pat. No. 5,641,484

U.S. Pat. No. 5,643,567

U.S. Pat. No. 5,651,964

U.S. Pat. No. 5,658,776

U.S. Pat. No. 5,677,178

U.S. Pat. No. 5,736,387

U.S. Pat. No. 5,776,743

U.S. Pat. No. 5,801,029

U.S. Pat. No. 5,814,315

U.S. Pat. No. 5,837,484

U.S. Pat. No. 5,846,945

U.S. Pat. No. 5,851,818

U.S. Pat. No. 5,856,181

U.S. Pat. No. 5,922,688

U.S. Pat. No. 5,948,885

U.S. Pat. No. 5,972,706

Baichwal and Sugden, In: Gene Transfer, Kucherlapati (ed.), New York, Plenum Press, 117-148, 1986.

Benvenisty and Neshif, Proc. Natl. Acad Sci. USA, 83(24):9551-9555, 1986.

Blomer et al., J Virol., 71(9):6641-6649, 1997.

Campisi and Medrano, J Cell Physiol., 114(1):53-60, 1983.

Carter, Curr. Opin. Biotechnol., 3:533-539, 1992.

Carter, Handbook of Parvoviruses, 1:169-228, 1990.

Chan, et al., 193(3):1152-58, 1993.

Chang et al., Hepatology, 14:134A, 1991.

Chatterjee et al., Ann. NY Acad. Sci., 770:79-90, 1995.

Chen and Okayama, Mol. Cell Biol., 7(8):2745-2752, 1987.

Coffin, In: Virology, Fields et al., eds., Raven Press, New York, pp. 1437-1500, 1990.

Cohen et al., FEBS Lett., 1997, 410, 3-10

Couch et al., Am. Rev. Resp. Dis., 88:394-403, 1963.

Coupar et al., Gene, 68:1-10, 1988.

DeCaprio, et al.,Cell, 54:275-283, 1988.

Du et al., Gene Therapy, 3:254261, 1996.

Dubensky et al., Proc. Nat'l Acad Sci. USA, 81:7529-7533, 1984.

European Application EPO 0273085

Fechheimer et al., Proc. Nat'l Acad. Sci. USA, 84:8463-8467, 1987.

Ferkol et al., FASEB J, 7:1081-1091, 1993.

Figge, et al.,J Virology, 62:(5)1814-18, 1988.

Flotte et al., Am. J. Respir. Cell Mol. Biol. 7:349-356, 1992

Flotte et al., Gene Therapy, 2:357-362, 1995

Fraley et al., Proc. Nat'l Acad Sci. USA, 76:3348-3352, 1979.

Franke et al., Science, 1997, 275, 665-668

Friedmann, Science, 244:1275-1281, 1989.

Gao and Huang, Biophysical Research Communications, 179:(1)280-85, 1991.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Gomez-Foix et al., J. Biol. Chem., 267:25129-25134, 1992.

Gopal, Mol. Cell Biol., 5:1188-1190, 1985.

Graham and van der Eb, Virology, 52:456-467, 1973.

Grunhaus et al.., Seminar in Virology, 200(2):535-546, 1992.

Haley, et al., Proc. Natl. Acad Sci. USA, 81:5734-38, 1984.

Harland and Weintraub, J. Cell Biol., 101: 1094-1099, 1985.

Harlow, et al., J. of Virology, 55(3):533-46, 1985.

Hermonat and Muzycska, Proc. Nat'l Acad. Sci. USA, 81:6466-6470, 1984.

Hersdorffer et al., DNA Cell Biol., 9:713-723, 1990.

Herz and Gerard, Proc. Nat'l Acad. Sci. USA, 90:2812-2816, 1993.

Horwich et al., Virol., 64:642-650, 1990.

Hoveling, et al., J. Virology, 105:537-50, 1980.

Hung, et al., Gene, 159:65-71, 1995.

Kaneda et al., Science, 243:375-378, 1989.

Kato et al, J. Biol. Chem., 266:3361-3364, 1991.

Klein et al.,Nature, 327:70-73, 1987.

Konishi et al., Biochem. Biophys. Res. Commun., 216, 526-534, 1995.

Kotin, Hum. Gene Ther., 5:793-801, 1994.

Le Gal La Salle et al., Science, 259:988-990, 1993.

Levrero et al., Gene, 101:195-202, 1991.

Mann et al., Cell, 33:153-159, 1983.

Markowitz et al., J. Virol., 62:1120-1124, 1988.

Meier et al., J. Biol. Chem., 1997, 272, 30491-30497.

Muller, et al., Nature, 299:640-44, 1982.

Muzyczka, Curr Top. Microbiol. Immunol., 158:92-129, 1992.

Mymryk, Oncogene, 13:1581-1589,1996.

Nabel, et al., Science, 249:1285-88, 1990.

Naldini et al., Science, 272(5259):263-267, 1996.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau, et al., Biology Cell, 47:121-130, 1983.

Nicolau, et al., Methods in Enzymology, 149:157-177, 1987.

Paskind et al., Virology, 67:242-248, 1975.

PCT Application PCT/US92/07290

PCT Application PCT/US95/04738

PCT Application PCT/US95/04738

PCT Application PCT/US95/04738

PCT Application PCT/US96/15388

PCT Application PCT/US97/06066

PCT Application PCT/US97/12544

PCT Application PCT/US97/12544

Perales et al., Proc. Nat'l Acad Sci. USA, 91:4086-4090, 1994.

Physicians Desk Reference

Potter et al., Proc. Nat'l Acad. Sci. USA, 81:7161-7165,1984.

Pozzatti, et al., Science, 232:223-27, 1986.

Pozzatti et al., Mol. Cell Biol., 8(7):2984-88, 1988.

Ragot et al., Nature, 361:647-650, 1993.

Ravindranath and Morton, Intern. Rev. Immunol., 7: 303-329, 1991.

Remington's Pharmaceutical Sciences, 15^{th} ed., pages 1035-1038 and 1570-1580, Mack

Publishing Company, Easton, Pa., 1980.

Renan, Radiother. Oncol., 19:197-218, 1990.

Rich et al., Hum. Gene Ther., 4:461-476, 1993.

Ridgeway, In: Vectors: A survey of molecular cloning vectors and their uses, Stoneham: Butterworth, pp. 467-492, 1988.

Rippe et al., Mol. Cell Biol., 10:689-695, 1990.

Rosenberg et al., Ann Surg. 210(4):474-548, 1989

Rosenfeld et al., Cell, 68:143-155, 1992.

Rosenfeld et al., Science, 252:431-434, 1991.

Roux et al., Proc. Nat'l Acad. Sci. USA, 86:9079-9083, 1989.

Ruley, Nature, 304:602-06, 1983.

Rustgi, et al., Nature, 352:541-44, 1991.

Smith and Rutledge, Natl. Cancer Inst. Monogr., 42:141-143, 1975.

Stratford-Perricaudet and Perricaudet, In: Human Gene Transfer, Eds, Cohen-Haguenauer and Boiron, John Libbey Eurotext, France, 51-61, 1991.

Stratford-Perricaudet et al., Hum. Gene. Ther., 1:241-256, 1990.

Temin, In: Gene Transfer, Kucherlapati (ed.), New York: Plenum Press, pp. 149-188, 1986.

Top et al.,J. Infect. Dis., 124:155-160, 1971.

Tur-Kaspa et al., Mol. Cell Biol., 6:716-718, 1986.

Varmus et al., Cell, 25:23-36, 1981.

Wagner et al., Proc. Nat'l Acad. Sci. USA 87(9):3410-3414, 1990.

Walker et al., Biochem. J., 1998, 331, 299-308

Wallich, et al., Nature, 315:301-05, 1985.

Weinberg, Science, 230:770-76, 1985.

Whyte, et al., Nature, 334:124-29, 1988.

WO 96/17947 June, 1996.

Wu and Wu, J. Biol. Chem., 262:4429-4432, 1987.

Wu and Wu, Biochemistry, 27:887-892, 1988.

Yang et al., Proc Nat'l Acad. Sci. USA, 87:9568-9572, 1990.

Young et al., N Engl J Med. 7:299(23):1261-1266, 1978.

Yu, et al., Mol. Cell. Bio., 11(3):1745-1750, 1991.

Yu, et al., Cancer Research, 53:891-98, 1993.

Yu, et al., Oncogene, 11:1383-88, 1995.

Yu, et al., Oncogene 7:2263-70, 1992.

Zelenin et al., FEBS Lett., 280:94-96, 1991.

Zufferey et al., Nat Biotechnol, 15(9):871-875, 1997.

What is claimed is:

1. A method of inactivating Akt in a cell comprising contacting the cell with an E1A protein, peptide, or polypeptide.

2. The method of claim 1, wherein the cell is a cell that overexpresses activated Akt.

3. The method of claim 1, wherein the cell is further characterized by p38 inactivation.

4. The method of claim 3, wherein contacting of the cell with the E1A protein, peptide, or polypeptide results in p38 activation.

5. The method of claim 1, wherein the cell is comprised in vitro.

6. The method of claim 1, wherein the cell is comprised in vivo.

7. The method of claim 1, wherein the cell is comprised in an a animal.

8. The method of claim 1, wherein the cell is a cancer cell.

9. The method of claim 1, wherein the cell is a breast cancer cell, an ovarian cancer cell, a pancreatic cancer cell, a prostate cancer cell, a colon cancer cell, a brain cancer cell, or a rectal cancer cell.

10. The method of claim 8, wherein the cancer cell is comprised in an animal.

11. The method of claim 10, wherein the animal is human.

12. The method of claim 10, further defined as a method of treating or preventing cancer.

13. The method of claim 12, wherein transformation and or growth of the cell is suppressed.

14. The method of claim 12, wherein apoptosis is induced in the cell.

15. The method of claim 1, wherein the contacting the cell with the E1A protein, peptide, or polypeptide comprises providing a nucleic acid encoding the E1A protein, peptide, or polypeptide and expressing the E1A protein, peptide, or polypeptide.

16. The method of claim 15, wherein the E1A protein, peptide, or polypeptide is encoded by a vector.

17. The method of claim 16, wherein the vector is a viral vector.

18. The method of claim 1, wherein the E1A protein, peptide, or polypeptide or a nucleic acid encoding the E1A protein, peptide, or polypeptide is comprised in a liposome.

19. The method of claim 1, wherein the contacting comprises administering the E1A protein, peptide, or polypeptide or a nucleic acid encoding the E1A protein, peptide, or polypeptide to an animal.

20. The method of claim 19, wherein the E1A protein, peptide, or polypeptide or nucleic acid encoding the E1A protein, peptide, or polypeptide is administered by injection.

21. The method of claim 19, wherein the E1A protein, peptide, or polypeptide or nucleic acid encoding the E1A protein, peptide, or polypeptide is administered systemically.

22. The method of claim 1, further comprising contacting the cell with a chemotherapeutic agent.

23. The method of claim 22, wherein the E1A protein, peptide, or polypeptide or nucleic acid encoding the E1A protein, peptide, or polypeptide is administered before or after administration of the chemotherapeutic agent.

24. A method of activating p38 in a cell comprising contacting the cell with an E1A protein, peptide, or polypeptide.

25. The method of claim 24, wherein the cell is a cell that underexpresses activated p38.

26. The method of claim 24, wherein the cell is further characterized by Akt activation.

27. The method of claim 26, wherein contacting of the cell with E1A protein, peptide, or polypeptide results in Akt inactivation.

28. The method of claim 24, wherein the cell is comprised in vitro.

29. The method of claim 24, wherein the cell is comprised in vivo.

30. The method of claim 24, wherein the cell is comprised in an a animal.

31. The method of claim 24, wherein the cell is a cancer cell.

32. The method of claim 24, wherein the cell is a breast cancer cell, an ovarian cancer cell, a pancreatic cancer cell, a prostate cancer cell, a colon cancer cell, a brain cancer cell, or a rectal cancer cell.

33. The method of claim 31, wherein the cancer cell is comprised in an animal.

34. The method of claim 33, wherein the animal is human.

35. The method of claim 33, further defined as a method of treating or preventing cancer.

36. The method of claim 35, wherein transformation and or growth of the cell is suppressed.

37. The method of claim 35, wherein apoptosis is induced in the cell.

38. The method of claim 24, wherein the contacting the cell with the E1A protein, peptide, or polypeptide comprises providing a nucleic acid encoding the E1A protein, peptide, or polypeptide and expressing the E1A protein, peptide, or polypeptide.

39. The method of claim 38, wherein the E1A protein, peptide, or polypeptide is encoded by a vector.

40. The method of claim 39, wherein the vector is a viral vector.

41. The method of claim 24, wherein the E1A protein, peptide, or polypeptide or a nucleic acid encoding the E1A protein, peptide, or polypeptide is comprised in a liposome.

42. The method of claim 24, wherein the contacting comprises administering the E1A protein, peptide, or polypeptide or a nucleic acid encoding the E1A protein, peptide, or polypeptide to an animal.

43. The method of claim 42, wherein the E1A protein, peptide, or polypeptide or nucleic acid encoding the E1A protein, peptide, or polypeptide is administered by injection.

44. The method of claim 42, wherein the E1A protein, peptide, or polypeptide or nucleic acid encoding the E1A protein, peptide, or polypeptide is administered systemically.

45. The method of claim 24, further comprising contacting the cell with a chemotherapeutic agent.

46. The method of claim 45, wherein the E1A protein, peptide, or polypeptide or nucleic acid encoding the E1A protein, peptide, or polypeptide is administered before or after administration of the chemotherapeutic agent.

47. A method of downregulating phospho-Akt expression and upregulating phospho-p38 expression in a cell comprising contacting the cell with an E1A protein, peptide, or polypeptide.

48. The method of claim 47, wherein the cell is a cancer cell.
